(19) United States
(12) Patent Application Publication
(10) Pub. No.: US 2024/0036059 A1
(43) Pub. Date: Feb. 1, 2024

(54) METHOD FOR DETERMINING WHETHER A SUBJECT IS AT RISK OF DEVELOPING AN ANEMIA AND/OR A METABOLIC CONDITION RELATED TO THE NUTRITIONAL STATE AND/OR FLUID BALANCE OF THE BLOOD

(71) Applicant: Nightingale Health Oyj, Helsinki (FI)

(72) Inventors: Heli Julkunen, Helsinki (FI); Peter Würtz, Helsinki (FI)

(21) Appl. No.: 18/039,883

(22) PCT Filed: Dec. 2, 2021
(86) PCT No.: PCT/FI2021/050837

§ 371 Date: Jun. 1, 2023

(30) Foreign Application Priority Data
Dec. 2, 2020 (FI) 20206239

(51) Int. Cl. G01N 33/68; G01N 24/08; G01N 33/92

(52) U.S. Cl. CPC G01N 33/6893; G01N 24/08; G01N 33/92; G01N 2800/04; G01N 2800/50

(57) ABSTRACT

A method for determining whether a subject is at risk of developing an anemia and/or a metabolic condition related to the nutritional state and/or fluid balance of the blood is disclosed.

### PRIORITY CLAIM

This application is the National Stage entry under 35 U.S.C. § 371 of International Application No. PCT/FI2021/050837, filed on Dec. 2, 2021, published on Jun. 9, 2022 under Publication Number WO 2022/117923 A1, which claims the benefit of priority under 35 U.S.C. § 119 of Finnish Patent Application Number FI20206239 filed on Dec. 2, 2020, the entireties of which are herein incorporated by reference.

### TECHNICAL FIELD

The present disclosure relates generally to a method for determining whether a subject is at risk of developing an anemia and/or a metabolic condition related to the nutritional state and/or fluid balance of the blood.

### BACKGROUND

Anemias and metabolic conditions of the blood are disorders affecting oxygen transport and overall blood hemostasis. These disorders can also reflect nutritional state and fluid balance of the blood, including vitamin deficiencies, impaired mineral metabolism, acid-base imbalance, as well as volume depletion and fluid overload. These disorders are frequent causes of healthcare encounters and hospitalisation. They often cause fatigue, weakness, and shortness of breath. They can also cause more long-term health problems such as organ damage, including enlarged heart, heart failure and other heart problems. Impaired mineral balance and electrolyte balance can also lead to muscle weakness, cramps and constipation. These metabolic conditions of the blood can further impair the innate and acquired immune responses, and hereby increase susceptibility to other more severe diseases.

Fortunately, there are effective treatments available for many forms of anemias and metabolic conditions related to the nutritional state or fluid balance of the blood if the disorders are identified early. Accurate identification of individuals at high risk for developing these disorders could help to avoid them of becoming overt and/or symptomatic. Novel tools for prediction of the risk for developing an anemia and/or a metabolic condition related to the nutritional state and/or fluid balance of the blood, could also benefit screening efforts, so preventative efforts and treatments can be targeted to those individuals who may need it the most.

There may also be a need for biomarkers that are predictive of the future risk for specific types of anemias and other metabolic conditions related to the nutritional state and fluid balance of the blood, such as iron deficiency anemia, other anemias, vitamin B and vitamin D deficiencies, disorders of mineral metabolism, volume depletion, other disorders of fluid, electrolyte and acid-base balance.

### SUMMARY

A method for determining whether a subject is at risk of developing an anemia and/or a metabolic condition related to the nutritional state and/or fluid balance of the blood is disclosed. The method may comprise determining in a biological sample obtained from the subject a quantitative value of at least one biomarker of the following:
albumin,
glycoprotein acetyls,
ratio of docosahexaenoic acid to total fatty acids,
ratio of linoleic acid to total fatty acids,
ratio of monounsaturated fatty acids and/or of oleic acid to total fatty acids,
ratio of omega-3 fatty acids to total fatty acids,
ratio of omega-6 fatty acids to total fatty acids,
fatty acid degree of unsaturation,
docosahexaenoic acid,
linoleic acid,
omega-3 fatty acids,
omega-6 fatty acids,
citrate,
pyruvate,
alanine,
glutamine,
histidine,
leucine,
phenylalanine,
valine; and
comparing the quantitative value(s) of the at least one biomarker to a control sample or to a control value;
wherein an increase or a decrease in the quantitative value(s) of at least one biomarker, when compared to the control sample or to the control value, is/are indicative of the subject having an increased risk of developing the anemia and/or the metabolic condition related to the nutritional state and/or fluid balance of the blood.

### BRIEF DESCRIPTION OF THE DRAWINGS

The accompanying drawings, which are included to provide a further understanding of the embodiments and constitute a part of this specification, illustrate various embodiments. In the drawings:

FIG. 1a shows the relation of baseline concentrations of 20 blood biomarkers to future development of Any Anemia and/or Any Metabolic Condition Related to the Nutritional State and/or Fluid Balance of the Blood (defined as the combined endpoint of any ICD-10 diagnoses within D50-D64, E50-E64 and E70-E88 except E78; here termed “Any Anemia and/or Any Metabolic Condition Related to the Nutritional State and/or Fluid Balance of the Blood”), when the biomarker concentrations are analysed in absolute concentrations and in quintiles of biomarker concentrations. Results are based on plasma samples from approximately 115,000 generally healthy individuals from the UK Biobank.

FIG. 1b shows the cumulative risk for “Any Anemia and/or Any Metabolic Condition Related to the Nutritional State and/or Fluid Balance of the Blood” during follow-up for the lowest, middle, and highest quintiles of the 20 blood biomarker concentrations.

FIG. 2a shows the relation of the baseline concentrations of the 20 blood biomarkers to future development of 11 different anemias and/or metabolic conditions related to the nutritional state and/or fluid balance of the blood (defined by ICD-10 3-character diagnoses), in the form of a heatmap. The results demonstrate that the 11 different metabolic conditions related to the nutritional state and/or fluid balance of the blood all have highly similar associations with the 20 biomarkers measured by nuclear magnetic resonance (NMR) spectroscopy of plasma samples from generally healthy humans.

FIG. 2b shows the consistency of the biomarker associations with the 11 different anemias and/or metabolic conditions related to the nutritional state and/or fluid balance of the blood, in comparison to the direction of corresponding biomarker associations with “Any Anemia and/or Any Metabolic Condition Related to the Nutritional State and/or Fluid Balance of the Blood”.

FIG. 3a shows the relation of baseline biomarker levels to the future development of 18 specific anemias and/or metabolic conditions related to the nutritional state and/or fluid balance of the blood (defined by ICD-10 4-character diagnoses) in the form of a heatmap. The results demonstrate that the specific anemias and/or metabolic conditions related to the nutritional state and/or fluid balance of the blood defined by 4-character ICD-10 codes all have highly similar associations with a broad panel of biomarkers measured by NMR spectroscopy of plasma samples from generally healthy humans.

FIG. 3b shows the consistency of the biomarker associations with specific anemias and/or metabolic conditions related to the nutritional state and/or fluid balance of the blood (defined by ICD-10 4-character diagnoses), in comparison to direction of the association with “Any Anemia and/or Any Metabolic Condition Related to the Nutritional State and/or Fluid Balance of the Blood”.

FIGS. 4a-f show the relation of baseline biomarker levels to the future development of 11 different anemias and/or metabolic conditions related to the nutritional state and/or fluid balance of the blood (defined by ICD-10 3-character diagnoses), in the form of foresplots of the hazard ratios for incident disease onset.

FIG. 5 shows an example of the relation of multi-biomarker scores to the risk of “Any Anemia and/or Any Metabolic Condition Related to the Nutritional State and/or Fluid Balance of the Blood”. Selected examples of multi-biomarker scores are shown to illustrate the improved prediction attained by multi-biomarker scores as compared to individual biomarkers.

FIG. 6a shows an intended use case for a multi-biomarker score to predict the risk for developing iron deficiency anemia among initially healthy humans.

FIG. 6b shows that the prediction of the risk for developing iron deficiency anemia works for people with different demographics and risk factor profiles, with substantially stronger results for short-term risk prediction.

FIG. 7a shows an intended use case for a multi-biomarker score to predict the risk for developing other anemias among initially healthy humans.

FIG. 7b shows that the prediction of the risk for developing other anemias works for people with different demographics and risk factor profiles, with substantially stronger results for short-term risk prediction.

FIG. 8a shows an intended use case for a multi-biomarker score to predict the risk for developing deficiency of other B group vitamins among initially healthy humans.

FIG. 8b shows that the prediction of the risk for developing deficiency of other B group vitamins works for people with different demographics and risk factor profiles, with substantially stronger results for short-term risk prediction.

FIG. 9a shows an intended use case for a multi-biomarker score to predict the risk for developing vitamin D deficiency among initially healthy humans.

FIG. 9b shows that the prediction of the risk for developing vitamin D deficiency works for people with different demographics and risk factor profiles, with substantially stronger results for short-term risk prediction.

FIG. 10a shows an intended use case for a multi-biomarker score to predict the risk for developing disorders of mineral metabolism among initially healthy humans.

FIG. 10b shows that the prediction of the risk for developing disorders of mineral metabolism works for people with different demographics and risk factor profiles, with substantially stronger results for short-term risk prediction.

FIG. 11a shows an intended use case for a multi-biomarker score to predict the risk for developing volume depletion among initially healthy humans.

FIG. 11b shows that the prediction of the risk for developing volume depletion works for people with different demographics and risk factor profiles, with substantially stronger results for short-term risk prediction.

FIG. 12a shows an intended use case for a multi-biomarker score to predict the risk for developing other disorders of fluid, electrolyte and acid-base balance among initially healthy humans.

FIG. 12b shows that the prediction of the risk for developing other disorders of fluid, electrolyte and acid-base balance works for people with different demographics and risk factor profiles, with substantially stronger results for short-term risk prediction.

### DETAILED DESCRIPTION

A method for determining whether a subject is at risk of developing an anemia and/or a metabolic condition related to the nutritional state and/or fluid balance of the blood is disclosed. The anemia and/or the metabolic condition related to the nutritional state and/or fluid balance of the blood may be an anemia or other metabolic condition related to the nutritional state and/or fluid balance of the blood, such as a vitamin deficiency, impaired mineral metabolism, acid-base imbalance, and/or volume depletion and fluid overload.

The method may comprise determining in a biological sample obtained from the subject a quantitative value of at least one biomarker of the following:
albumin,
glycoprotein acetyls,
ratio of docosahexaenoic acid to total fatty acids,
ratio of linoleic acid to total fatty acids,
ratio of monounsaturated fatty acids and/or of oleic acid to total fatty acids,
ratio of omega-3 fatty acids to total fatty acids,
ratio of omega-6 fatty acids to total fatty acids,
fatty acid degree of unsaturation,
docosahexaenoic acid,
linoleic acid,
omega-3 fatty acids,
omega-6 fatty acids,
citrate,
pyruvate,
alanine,
glutamine,
histidine,
leucine,
phenylalanine,
valine; and
and comparing the quantitative value(s) of the at least one biomarker to a control sample or to a control value;
wherein an increase or a decrease in the quantitative value(s) of the at least one biomarker, when compared to the control sample or to the control value, is/are indicative of the subject having an increased risk of developing the anemia and/or the metabolic condition related to the nutritional state and/or fluid balance of the blood.

Various blood biomarkers may be useful for predicting whether an individual person is at elevated risk of developing a broad range of anemias and/or metabolic conditions related to the nutritional state and/or fluid balance of the blood. Such biomarkers may be measured from biological samples, for example from blood samples or related biological fluids.

Biomarkers predictive of hospitalization from an anemia and/or a metabolic condition related to the nutritional state and/or fluid balance of the blood could help to enable more effective screening and better targeted preventative treatments, such as dietary and supplement interventions according to the metabolic profile.

In an embodiment, the method comprises determining a quantitative value of albumin.

In an embodiment, the method comprises determining a quantitative value of glycoprotein acetyls.

In an embodiment, the method comprises determining a quantitative value of fatty acid degree of unsaturation.

In an embodiment, the method comprises determining a quantitative value of ratio of docosahexaenoic acid to total fatty acids.

In an embodiment, the method comprises determining a quantitative value of ratio of linoleic acid to total fatty acids.

In an embodiment, the method comprises determining a quantitative value of ratio of monounsaturated fatty acids and/or oleic acid to total fatty acids.

In an embodiment, the method comprises determining a quantitative value of ratio of omega-3 fatty acids to total fatty acids.

In an embodiment, the method comprises determining a quantitative value of ratio of omega-6 fatty acids to total fatty acids.

In an embodiment, the method comprises determining a quantitative value of docosahexaenoic acid.

In an embodiment, the method comprises determining a quantitative value of linoleic acid.

In an embodiment, the method comprises determining a quantitative value of omega-3 fatty acids.

In an embodiment, the method comprises determining a quantitative value of omega-6 fatty acids.

In an embodiment, the method comprises determining a quantitative value of citrate.

In an embodiment, the method comprises determining a quantitative value of pyruvate.

In an embodiment, the method comprises determining a quantitative value of alanine.

In an embodiment, the method comprises determining a quantitative value of glutamine.

In an embodiment, the method comprises determining a quantitative value of histidine.

In an embodiment, the method comprises determining a quantitative value of leucine.

In an embodiment, the method comprises determining a quantitative value of phenylalanine.

In an embodiment, the method comprises determining a quantitative value of valine.

The metabolic biomarker(s) described in this specification have been found to be significantly different, i.e. their quantitative values (such as amount and/or concentration) have been found to be significantly higher or lower, for subjects who later developed an anemia and/or a metabolic condition related to the nutritional state and/or fluid balance of the blood. The biomarkers may be detected and quantified from blood, serum, or plasma, dry blood spots, or other suitable biological sample, and may be used to determine the risk of developing an anemia and/or a metabolic condition related to the nutritional state and/or fluid balance of the blood, either alone or in combination with other biomarkers.

Furthermore, the biomarker(s) may significantly improve the possibility of identifying subjects at risk for an anemia and/or a metabolic condition related to the nutritional state and/or fluid balance of the blood, even when accounting for established risk factors that may currently be used for screening and risk prediction, such as age, poor diet, smoking status, adiposity measures such as body mass index (BMI), high blood pressure, high cholesterol, family history, genetic risk and/or prior medical history of certain diseases, such as autoimmune diseases and/or diseases of the liver, kidney or thyroid. The biomarkers described in this specification, alone or as a risk score (such as a multi-biomarker risk score), hazard ratio, odds ratio, and/or predicted absolute or relative risk or in combination with other risk factors and tests, may improve prediction on top of or even replace the need for other tests or measures. This may include improving prediction accuracy or replacing the need for other analyses such as complete blood count measurements (CBC; including concentrations of hemoglobin and hematocrit, counts of red blood cells, white blood cells and platelets, and mean corpuscular volume), hemoglobin electrophoresis, reticulocyte count, test for the level of iron in the blood, such as serum iron, serum ferritin, transferrin level or total iron-binding capacity, serum electrolyte, and/or vitamin tests. The biomarkers or the risk score, hazard ratio, odds ratio, and/or predicted absolute or relative risk according to one or more embodiments described in this specification may thus allow for efficiently assessing the risk for future development of an anemia and/or a metabolic condition related to the nutritional state and/or fluid balance of the blood, also in conditions in which other risk factor measures are not as feasible.

In an embodiment, the method is a method for determining whether the subject is at risk of developing an anemia and/or a metabolic condition related to the nutritional state and/or fluid balance of the blood.

The method may comprise determining in the biological sample quantitative values of a plurality of the biomarkers, such as two, three, four, five or more of the biomarkers. For example, the plurality of the biomarkers may comprise 2, 3, 4, 5, 6, 7, 8, 9, 10, 11, 12, 13, 14, 15, 16, 17, 18, 19, or 20 of the biomarkers (i.e. at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, or all of the biomarkers). The term “plurality of the biomarkers” may thus, within this specification, be understood as referring to any number (above one) of the biomarkers. The term “plurality of the biomarkers” may thus be understood as referring to any number (above one) and/or combination or subset of the biomarkers described in this specification. Determining the plurality of the biomarkers may increase the accuracy of the prediction of whether the subject is at risk of developing an anemia and/or a metabolic condition related to the nutritional state and/or fluid balance of the blood. In general, it may be that the higher the number of the biomarkers, the more accurate or predictive the method. However, even a single biomarker described in this specification may allow for or assist in determining whether the subject is at risk of developing the anemia and/or the metabolic condition related to the nutritional state and/or fluid balance of the blood. The plurality of the biomarkers may be measured from the same biological sample or from separate biological samples and using the same analytical method or different analytical methods. In an embodiment, the plurality of biomarkers may be a panel of a plurality of biomarkers.

In the context of this specification, the wording “comparing the quantitative value(s) of the biomarker(s) to a control sample or to a control value(s)” may be understood, as a skilled person would, as referring to comparing the quantitative value or values of the biomarker or biomarkers, to a control sample or to a control value(s) either individually or as a plurality of biomarkers (e.g. when a risk score is calculated from the quantitative values of a plurality of biomarkers), depending e.g. on whether the quantitative value of a single (individual) biomarker or the quantitative values of a plurality of biomarkers are determined.

In an embodiment, the method may comprise determining in the biological sample obtained from the subject a quantitative value of the following biomarkers:
albumin,
glycoprotein acetyls,
ratio of docosahexaenoic acid to total fatty acids,
ratio of linoleic acid to total fatty acids,
ratio of monounsaturated fatty acids and/or of oleic acid to total fatty acids,
ratio of omega-3 fatty acids to total fatty acids,
ratio of omega-6 fatty acids to total fatty acids,
fatty acid degree of unsaturation,
docosahexaenoic acid,
linoleic acid,
omega-3 fatty acids,
omega-6 fatty acids,
citrate,
pyruvate,
alanine,
glutamine,
histidine,
leucine,
phenylalanine, and
valine; and
comparing the quantitative value(s) of the biomarkers to a control sample or to a control value(s);
wherein an increase or a decrease in the quantitative value(s) of the biomarkers, when compared to the control sample or to the control value, is/are indicative of the subject having an increased risk of developing the anemia and/or the metabolic condition related to the nutritional state and/or fluid balance of the blood.

In an embodiment, the at least one biomarker comprises or is glycoprotein acetyls. The method may further comprise determining a quantitative value of at least one of the other biomarkers described in this specification.

The subject may be human. The human may be healthy or have an existing disease, such as an existing metabolic condition related to the nutritional state and/or fluid balance of the blood. Specifically, the human may have an already existing form of an anemia and/or a metabolic condition related to the nutritional state and/or fluid balance of the blood, and the risk for developing a more severe form of the disease/condition and/or other anemia and/or metabolic condition related to the nutritional state and/or fluid balance of the blood may be determined and/or calculated. The subject may, additionally or alternatively, be an animal, such as a mammal, for example, a non-human primate, a dog, a cat, a horse, or a rodent.

In the context of this specification, the term “biomarker” may refer to a biomarker, for example a chemical or molecular marker, that may be found to be associated with a disease or a condition or the risk of having or developing thereof. It does not necessarily refer to a biomarker that would be statistically fully validated as having a specific effectiveness in a clinical setting. The biomarker may be a metabolite, a compound, a lipid, a protein, a moiety, a functional group, a composition, a combination of two or more metabolites and/or compounds, a (measurable or measured) quantity thereof, a ratio or other value derived thereof, or in principle any measurement reflecting a chemical and/or biological component that may be found associated with a disease or condition or the risk of having or developing thereof. The biomarkers and any combinations thereof, optionally in combination with further analyses and/or measures, may be used to measure a biological process indicative of the risk for developing an anemia and/or a metabolic condition related to the nutritional state and/or fluid balance of the blood, such as an iron deficiency anemia, other anemia, vitamin deficiency, disorder of mineral metabolism, volume depletion, and/or other disorder of fluid, electrolyte and/or acid-base balance.

The disease or condition may refer to a disease or condition in the categories of anemias and/or metabolic conditions related to the nutritional state and/or fluid balance of the blood or to a specific disease in these categories. In the context of this specification, the term “metabolic condition related to the nutritional state and/or fluid balance of the blood” may be understood as referring to a disease, disorder and/or condition of, or primarily affecting, blood and/or blood forming organs. This group of disorders includes, but is not limited to, various types of anemias and other metabolic conditions related to the nutritional state and/or fluid balance of the blood, such as an impaired mineral metabolism, acid-base imbalance, nutritional deficiencies and impaired fluid balance. The disease or condition can be acute or chronic. The signs and symptoms of these diseases may vary from mild to severe or disabling or potentially life-threatening, depending on factors such as age and/or overall health of the subject.

The biomarker associations may be similar for the different anemias and/or metabolic conditions related to the nutritional state and/or fluid balance of the blood. Therefore, the same individual biomarkers and combinations of biomarkers may be extended to also predict the risk for specific anemias and/or metabolic conditions related to the nutritional state and/or fluid balance of the blood. Examples of such specific anemias and/or metabolic conditions related to the nutritional state and/or fluid balance of the blood may include iron deficiency anemia, other anemias, vitamin deficiencies, disorders of mineral metabolism, volume depletion, other disorders of fluid, electrolyte and acid-base balance.

The anemias and/or the metabolic conditions related to the nutritional state and/or fluid balance of the blood described in this specification may be classified as follows. “ICD-10” may be understood as referring to the International Statistical Classification of Diseases and Related Health Problems 10th Revision (ICD-10)—WHO Version for 2019. Similar conditions classified or diagnosed by other disease classification systems than ICD-10, such as ICD-9 or ICD-11, may also apply.

The term “Any Anemia and/or Any Metabolic Condition Related to the Nutritional State and/or Fluid Balance of the Blood” may be understood as referring to any metabolic disorder, disease or condition related to the nutritional state and/or fluid balance of the blood, such as an anemia. Any Anemia and/or Any Metabolic Condition Related to the Nutritional State and/or Fluid Balance of the Blood may be understood as referring to any incident occurrence of ICD-10 diagnoses D50-D89, E50-E64 or E70-E88, except E78.

Specific anemias and/or metabolic conditions related to the nutritional state and/or fluid balance of the blood may be understood as referring to diseases and/or conditions classified within the 3-character ICD-10 diagnoses for anemias and/or metabolic conditions related to the nutritional state and/or fluid balance of the blood (D50, D51, D61, D63, D64, E53, E55, E61, E83, E86, E87) or as 4-character IDC-10 diagnoses within these 3-character ICD-10 diagnoses (D50.8, D50.9, D51.0, D51.9, D63.0, D64.9, E53.8, E55.9, E61.1, E83.3, E83.4, E83.5, E87.0, E87.1, E87.2, E87.5, E87.6, E87.7). The term “anemia and/or metabolic condition related to the nutritional state and/or fluid balance of the blood” may thus be understood as referring to and encompassing various different anemias and/or metabolic conditions related to the nutritional state and/or fluid balance of the blood.

In an embodiment, the anemia and/or the metabolic condition related to the nutritional state and/or fluid balance of the blood is a specific disease or condition, such as a specific disease or condition defined by a ICD-10 3-character code diagnosis/diagnoses and/or by a ICD-10 4-character code diagnosis/diagnoses described herein.

In an embodiment, the anemia and/or the metabolic condition related to the nutritional state and/or fluid balance of the blood is a condition of (one or more of) the following ICD-10 3-character diagnoses:
D50: Iron deficiency anemia
D51: Vitamin B12 deficiency anemia
D61: Other aplastic anemias and other bone marrow failure syndromes
D63: Anemia in chronic diseases classified elsewhere
D64: Other anemias
E53: Deficiency of other B group vitamins
E55: Vitamin D deficiency
E61: Deficiency of other nutrient elements
E83: Disorders of mineral metabolism
E86: Volume depletion
E87: Other disorders of fluid, electrolyte and acid-base balance

In an embodiment, the anemia and/or the metabolic condition related to the nutritional state and/or fluid balance of the blood is a condition of (one or more of) the following ICD-4-character diagnoses:
D50.8: Other iron deficiency anemias
D50.9: Iron deficiency anemia, unspecified
D51.0: Vitamin B12 deficiency anemia due to intrinsic factor deficiency
D61.9: Aplastic anemia, unspecified
D63.0: Anemia in neoplastic disease
E83.3: Disorders of phosphorus metabolism and phosphatases
D64.9: Anemia, unspecified
E53.8: Deficiency of other specified B group vitamins
E55.9: Vitamin D deficiency, unspecified
E61.1: Iron deficiency
E83.4: Disorders of magnesium metabolism
E83.5: Disorders of calcium metabolism
E87.0: Hyperosmolality and hypernatremia
E87.1: Hypo-osmolality and hyponatremia
E87.2: Acidosis
E87.5: Hyperkalemia
E87.6: Hypokalemia
E87.7: Fluid overload

In an embodiment, the anemia and/or the metabolic condition related to the nutritional state and/or fluid balance of the blood may comprise or be death from an anemia and/or a metabolic condition related to the nutritional state and/or fluid balance of the blood, such as a disease or condition denoted by or in a group of any of the ICD-10 codes listed above.

In an embodiment, the specific anemia and/or metabolic condition related to the nutritional state and/or fluid balance of the blood may comprise or be iron deficiency anemia (D50); vitamin B12 deficiency anemia (D51); other aplastic anemia or other bone marrow failure syndrome (D61); anemia in chronic diseases classified elsewhere (D63); other anemia (D64); deficiency of other B group vitamins (E53); vitamin D deficiency (E55); deficiency of other nutrient elements (E61); disorder of mineral metabolism (E83); volume depletion (E86); or other disorder of fluid, electrolyte and/or acid-base balance (E87).

In an embodiment, the anemia and/or metabolic condition related to the nutritional state and/or fluid balance of the blood may comprise or be other iron deficiency anemia (D50.8); iron deficiency anemia, unspecified (D50.9); vitamin B12 deficiency anemia due to intrinsic factor deficiency (D51.0); aplastic anemia, unspecified (D61.9); anemia in neoplastic disease (D63.0); disorder of phosphorus metabolism and/or phosphatases (E83.3); anemia, unspecified (D64.9); deficiency of other specified B group vitamins (E53.8; vitamin D deficiency, unspecified (E55.9); iron deficiency (E61.1); disorder of magnesium metabolism (E83.4); disorder of calcium metabolism (E83.5); hyperosmolality and/or hypernatremia (E87.0); hypo-osmolality and/or hyponatremia (E87.1); acidosis (E87.2); hyperkalemia (E87.5); and/or hypokalemia (E87.6).

In an embodiment, the anemia and/or the metabolic condition related to the nutritional state and/or fluid balance of the blood may comprise or be iron deficiency anemia, other anemia, deficiency of B group vitamins, vitamin D deficiency, disorder of mineral metabolism, volume depletion and/or other disorder of fluid, electrolyte and acid-base balance.

The method may further comprise determining whether the subject is at risk of developing the anemia and/or the metabolic condition related to the nutritional state and/or fluid balance of the blood using a risk score (such as a multi-biomarker risk score), hazard ratio, and/or predicted absolute or relative risk calculated on the basis of the quantitative value(s) of the at least one biomarker or of the plurality of the biomarkers.

An increase or a decrease in the risk score, hazard ratio, and/or predicted absolute risk and/or relative risk may be indicative of the subject having an increased risk of developing the anemia and/or the metabolic condition related to the nutritional state and/or fluid balance of the blood.

The risk score and/or hazard ratio and/or predicted absolute risk or relative risk may be calculated based on any plurality, combination or subset of biomarkers described in this specification.

The risk score and/or hazard ratio and/or predicted absolute risk or relative risk may be calculated e.g. as shown in the Examples below. For example, the plurality of biomarkers measured using a suitable method, for example with NMR spectroscopy, may be combined using regression algorithms and multivariate analyses and/or using machine learning analysis. Before regression analysis or machine learning, any missing values in the biomarkers may be imputed with the mean value of each biomarker for the dataset. A number of the biomarkers, for example five, that may be considered most associated with the onset of the disease or condition may be selected for use in the prediction model. Other modelling approaches may be used to calculate a risk score and/or hazard ratio and/or predicted absolute risk or relative risk based on a combination or subset of individual biomarkers, i.e. a plurality of the biomarkers.

The risk score may be calculated e.g. as a weighted sum of individual biomarkers, i.e. a plurality of the biomarkers. The weighted sum may be e.g. in the form of a multi-biomarker score defined as Σᵢ [symbol] βᵢ+cᵢ [symbol] +β₀; where i is the index of summation over individual biomarkers, β, is the weighted coefficient attributed to biomarker i, cᵢ is the blood concentration of biomarker i, and β₀ is an intercept term.

For example, the risk score can be defined as: β₁*concentration(glycoprotein acetyls) β₂*concentration(monounsaturated fatty acid ratio to total fatty acids)+β₃*concentration(albumin)+β₀, where β₁, β₂, β₃ are multipliers for each biomarker according to the association magnitude with risk of an anemia and/or a metabolic condition related to the nutritional state and/or fluid balance of the blood and β₀ is an intercept term. As a skilled person will understand, the biomarkers mentioned in this example may be replaced by any other biomarker(s) described in this specification. In general, the more biomarkers are included in the risk score, the stronger the predictive performance may become. When additional biomarkers are included in the risk score, the βi weights may change for all biomarkers according to the optimal combination for prediction of an anemia and/or a metabolic condition related to the nutritional state and/or fluid balance of the blood.

The risk score, hazard ratio, odds ratio, and/or predicted relative risk and/or absolute risk may be calculated on the basis of at least one further measure, for example a characteristic of the subject. Such characteristics may be determined (or may have been determined) prior to, simultaneously, or after the biological sample is obtained from the subject. As a skilled person will understand, some of the characteristics may be information collected e.g. using a questionnaire or clinical data collected earlier. Some of the characteristics may be determined (or may have been determined) by biochemical or clinical diagnostic measurements and/or medical diagnosis. Such characteristics could include, for example, one or more of age, height, weight, body mass index, race or ethnic group, smoking, and/or family history of anemias and/or metabolic conditions related to the nutritional state and/or fluid balance of the blood.

The method may further comprise administering a treatment to the subject at risk of developing an anemia and/or a metabolic condition related to the nutritional state and/or fluid balance of the blood to thereby treat the subject in order to prevent or treat the disease or condition in the subject.

The risk prediction for the anemia and/or the metabolic condition related to the nutritional state and/or fluid balance of the blood based on one or more of the biomarkers can be used to guide preventive efforts (such as diet, exercise, and/or supplement use), clinical screening frequency and/or pharmacological treatment decisions. For example, the information of the future risk for the anemia and/or the metabolic condition related to the nutritional state and/or fluid balance of the blood can be used for guiding preventative actions, such as the use of vitamin and/or mineral supplements, e.g. iron and folic acid supplements, coagulation factor medications and/or referral to a nutritionist and further medical examinations.

In the context of this specification, the term “albumin” may be understood as referring to serum albumin (often referred to as blood albumin). It is an albumin found in vertebrate blood. Albumin is a globular, water-soluble, un-glycosylated serum protein of approximate molecular weight of 65,000 Daltons. The measurement of albumin using NMR is described e.g. in publications by Kettunen et al., 2012, Nature Genetics 44, 269-276; Soininen et al., 2015, Circulation: Cardiovascular Genetics 8, 212-206 (DOI: Albumin may also be measured by various other methods, for example by clinical chemistry analyzers. Examples of such methods may include e.g. dye-binding methods such as bromocresol green and bromocresol purple.

In the context of this specification, the term “glycoprotein acetyls”, “glycoprotein acetylation”, or “GlycA” may refer to the abundance of circulating glycated proteins, and/or to a nuclear magnetic resonance spectroscopy (NMR) signal that represents the abundance of circulating glycated proteins, i.e. N-acetylated glycoproteins. Glycoprotein acetyls may include signals from a plurality of different glycoproteins, including e.g. alpha-1-acid glycoprotein, alpha-1 antitrypsin, haptoglobin, transferrin, and/or alpha-1 antichymotrypsin. In the scientific literature on cardiometabolic biomarkers, the terms “glycoprotein acetyls” or “GlycA” may commonly refer to the NMR signal of circulating glycated proteins (e.g. Ritchie et al, Cell Systems 2015 1(4):293-301; Connelly et al, J Transl Med. 2017; 15(1):219). Glycoprotein acetyls and a method for measuring them is described e.g. in Kettunen et al., 2018, Circ Genom Precis Med. 11:e002234 and Soininen et al., 2009, Analyst 134, 1781-1785. There may be benefits of using the NMR signal of glycoprotein acetyls for risk prediction above measurement of the individual proteins contributing to the NMR signal, for instance better analytical accuracy and stability over time, as well as lower costs of the measurement and the possibility to measure the NMR signal simultaneously with many other biomarkers.

In the context of this specification, the term “omega-3 fatty acids” may refer to total omega-3 fatty acids, i.e. the total omega-3 fatty acid amounts and/or concentrations, i.e. the sum of different omega-3 fatty acids. Omega-3 fatty acids are polyunsaturated fatty acids. In omega-3 fatty acids, the last double bond in the fatty acid chain is the third bond counting from the methyl end. Docosahexaenoic acid is an example of an omega-3 fatty acid.

In the context of this specification, the term “monounsaturated fatty acids” (MUFAs) may refer to total monounsaturated fatty acids, i.e. the total MUFA amounts and/or concentrations. Monounsaturated fatty acids may, alternatively, refer to oleic acid, which is the most abundant monounsaturated fatty acid in human serum. Monounsaturated fatty acids have one double bond in their fatty acid chain. The monounsaturated fatty acids may include omega-9 and omega-7 fatty acids. Oleic acid (18:1ω-9), palmitoleic acid (16:1ω-7) and cis-vaccenic acid (18:1ω-7) are examples of common monounsaturated fatty acids in human serum.

In one embodiment, the monounsaturated fatty acid may be oleic acid. Oleic acid is the most abundant monounsaturated fatty acid, and may therefore be considered as a good approximation for total monounsaturated fatty acids for risk prediction of the anemia and/or the metabolic condition related to the nutritional state and/or fluid balance of the blood.

In the context of this specification, the term “omega-6 fatty acids” may refer to total omega-6 fatty acids, i.e. the total omega-6 fatty acid amounts and/or concentrations, i.e. the sum of the amounts and/or concentrations of different omega-6 fatty acids. Omega-6 fatty acids are polyunsaturated fatty acids. In omega-6 fatty acids, the last double bond in the fatty acid chain is the sixth bond counting from the methyl end.

In one embodiment, the omega-6 fatty acid may be linoleic acid. Linoleic acid (18:2ω-6) is the most abundant type of omega-6 fatty acids, and may therefore be considered as a good approximation for total omega-6 fatty acids for risk prediction of the anemia and/or the metabolic condition related to the nutritional state and/or fluid balance of the blood.

For all fatty acid measures, including omega-3, omega-6, docosahexaenoic acid, linoleic acid, monounsaturated fatty acids, the fatty acid measures may include blood (or serum/plasma) free fatty acids, bound fatty acids and esterified fatty acids. Esterified fatty acids may, for example, be esterified to glycerol as in triglycerides, diglycerides, monoglycerides, or phosphoglycerides, or to cholesterol as in cholesterol esters.

In the context of this specification, the term “fatty acid degree of unsaturation” or “unsaturation” may be understood as referring to the number of double bonds in total fatty acids, for example the average number of double bonds in total fatty acids.

In the context of this specification, the term “citrate” may refer to the citrate molecule and/or citric acid, for example in blood, plasma or serum or related biofluids.

In the context of this specification, the term “pyruvate” may refer to the pyruvate molecule and/or pyruvic acid, for example in blood, plasma or serum or related biofluids.

In the context of this specification, the term “alanine” may refer to the alanine amino acid, for example in blood, plasma or serum or related biofluids.

In the context of this specification, the term “glutamine” may refer to the glutamine amino acid, for example in blood, plasma or serum or related biofluids.

In the context of this specification, the term “histidine” may refer to the histidine amino acid, for example in blood, plasma or serum or related biofluids.

In the context of this specification, the term “leucine” may refer to the leucine amino acid, for example in blood, plasma or serum or related biofluids.

In the context of this specification, the term “phenylalanine” may refer to the phenylalanine amino acid, for example in blood, plasma or serum or related biofluids.

In the context of this specification, the term “valine” may refer to the valine amino acid, for example in blood, plasma or serum or related biofluids.

In the context of this specification, the term “quantitative value” may refer to any quantitative value characterizing the amount and/or concentration of a biomarker. For example, it may be the amount or concentration of the biomarker in the biological sample, or it may be a signal derived from nuclear magnetic resonance spectroscopy (NMR) or other method suitable for detecting the biomarker in a quantitative manner. Such a signal may be indicative of or may correlate with the amount or concentration of the biomarker. It may also be a quantitative value calculated from one or more signals derived from NMR measurements or from other measurements. Quantitative values may, additionally or alternatively, be measured using a variety of techniques. Such methods may include mass spectrometry (MS), gas chromatography combined with MS, high performance liquid chromatography alone or combined with MS, immunoturbidimetric measurements, ultracentrifugation, ion mobility, enzymatic analyses, colorimetric or fluorometric analyses, immunoblot analysis, immunohistochemical methods (e.g. in situ methods based on antibody detection of metabolites), and immunoassays (e.g. ELISA). Examples of various methods are set out below. The method used to determine the quantitative value(s) in the subject may be the same method that is used to determine the quantitative value(s) in a control subject/control subjects or in a control sample/control samples.

The quantitative value, or the initial quantitative value, of the at least one biomarker, or the plurality of the biomarkers, may be measured using nuclear magnetic resonance (NMR) spectroscopy, for example ¹H-NMR. The at least one additional biomarker, or the plurality of the additional biomarkers, may also be measured using NMR. NMR may provide a particularly efficient and fast way to measure biomarkers, including a large number of biomarkers simultaneously, and can provide quantitative values for them. NMR also typically requires very little sample pre-treatment or preparation. The biomarkers measured with NMR can effectively be measured for large amounts of samples using an assay for blood (serum or plasma) NMR metabolomics previously published by Soininen et al., 2015, Circulation: Cardiovascular Genetics 8, 212-206 (DOI: 10.1161/CIRCGENETICS.114.000216); Soininen et al., 2009, Analyst 134, 1781-1785; and Würtz et al., 2017, American Journal of Epidemiology 186 (9), 1084-1096 (DOI: This provides data on 250 biomarkers per sample as described in detail in the above scientific papers.

In an embodiment, the (initial) quantitative value of the at least one biomarker is/are measured using nuclear magnetic resonance spectroscopy.

However, quantitative values for various biomarkers described in this specification may also be performed by techniques other than NMR. For example, mass spectrometry (MS), enzymatic methods, antibody-based detection methods, or other biochemical or chemical methods may be contemplated, depending on the biomarker.

For example, glycoprotein acetyls can be measured or approximated by immunoturbidimetric measurements of alpha-1-acid glycoprotein, haptoglobin, alpha-1-antitrypsin, and transferrin (e.g. as described in Ritchie et al., 2015, Cell Syst. 28; 1(4):293-301).

E.g. monounsaturated fatty acids and omega-3 fatty acids and omega-6 fatty acids can be quantified (i.e. their quantitative values may be determined) by serum total fatty acid composition using gas chromatography (for example, as described in Jula et al., 2005, Arterioscler Thromb Vasc Biol 25, 2152-2159).

In the context of this specification, the term “sample” or “biological sample” may refer to any biological sample obtained from a subject or a group or population of subjects. The sample may be fresh, frozen, or dry.

The biological sample may comprise or be, for example, a blood sample, a plasma sample, a serum sample, or a sample or fraction derived therefrom. The biological sample may be, for example, a fasting blood sample, a fasting plasma sample, a fasting serum sample, or a fraction obtainable therefrom. However, the biological sample does not necessarily have to be a fasting sample. The blood sample may be a venous blood sample.

The blood sample may be a dry blood sample. The dry blood sample may be a dried whole blood sample, a dried plasma sample, a dried serum sample, or a dried sample derived therefrom.

The method may comprise obtaining the biological sample from the subject prior to determining the quantitative value of the at least one biomarker. Taking a blood sample or a tissue sample of a subject or patient is a part of normal clinical practice. The collected blood or tissue sample can be prepared and serum or plasma can be separated using techniques well known to a skilled person. Methods for separating one or more fractions from biological samples, such as blood samples or tissue samples, are also available to a skilled person. The term “fraction” may, in the context of this specification, also refer to a portion or a component of the biological sample separated according to one or more physical properties, for instance solubility, hydrophilicity or hydrophobicity, density, or molecular size.

In the context of this specification, the term “control sample” may refer to a sample obtained from a subject and known not to suffer from the disease or condition or not being at risk of having or developing the disease or condition. The control sample may be matched. In an embodiment, the control sample may be a biological sample from a healthy individual or a generalized population of healthy individuals. The term “control value” may be understood as a value obtainable from the control sample and/or a quantitative value derivable therefrom. For example, it may be possible to calculate a threshold value from control samples and/or control values, above or below which the risk of developing the disease or condition is elevated. In other words, a value higher or lower (depending on the biomarker, risk score, hazard ratio, and/or predicted absolute risk or relative risk) than the threshold value may be indicative of the subject having an increased risk of developing the disease or condition.

An increase or a decrease in the quantitative value(s) of the at least one biomarker, or the plurality of the biomarkers, when compared to the control sample or to the control value, may be indicative of the subject having an increased risk of having or developing the disease or condition. Whether an increase or a decrease is indicative of the subject having an increased risk of developing the disease or condition, may depend on the biomarker.

A 1.2-fold, 1.5-fold, or for example 2-fold, or 3-fold, increase or a decrease in the quantitative value(s) of the at least one biomarker (or in an individual biomarker of the plurality of the biomarkers) when compared to the control sample or to the control value, may be indicative of the subject having an increased risk of developing the disease or condition.

In an embodiment, a decrease in the quantitative value of albumin, when compared to the control sample or to the control value, may be indicative of the subject having an increased risk of developing the anemia and/or the metabolic condition related to the nutritional state and/or fluid balance of the blood, such as an iron deficiency anemia, other anemia, deficiency of B group vitamins, vitamin D deficiency, disorder of mineral metabolism, volume depletion and/or other disorder of fluid, electrolyte and acid-base balance.

In an embodiment, an increase in the quantitative value of glycoprotein acetyls, when compared to the control sample or to the control value, may be indicative of the subject having an increased risk of developing the anemia and/or thew metabolic condition related to the nutritional state and/or fluid balance of the blood, such as an iron deficiency anemia, other anemia, deficiency of B group vitamins, vitamin D deficiency, disorder of mineral metabolism, volume depletion and/or other disorder of fluid, electrolyte and acid-base balance.

In an embodiment, a decrease in the quantitative value of docosahexaenoic acid ratio to total fatty acid, when compared to the control sample or to the control value, may be indicative of the subject having an increased risk of developing the anemia and/or the metabolic condition related to the nutritional state and/or fluid balance of the blood, such as an iron deficiency anemia, other anemia, deficiency of B group vitamins, vitamin D deficiency, disorder of mineral metabolism, volume depletion and/or other disorder of fluid, electrolyte and acid-base balance.

In an embodiment, a decrease in the quantitative value of linoleic acid ratio to total fatty acid, when compared to the control sample or to the control value, may be indicative of the subject having an increased risk of developing the anemia and/or the metabolic condition related to the nutritional state and/or fluid balance of the blood, such as an iron deficiency anemia, other anemia, deficiency of B group vitamins, vitamin D deficiency, disorder of mineral metabolism, volume depletion and/or other disorder of fluid, electrolyte and acid-base balance.

In an embodiment, an increase in the quantitative value of the ratio of monounsaturated fatty acid and/or of oleic acid to total fatty acids, when compared to the control sample or to the control value, may be indicative of the subject having an increased risk of developing the anemia and/or the metabolic condition related to the nutritional state and/or fluid balance of the blood, such as an iron deficiency anemia, other anemia, deficiency of B group vitamins, vitamin D deficiency, disorder of mineral metabolism, volume depletion and/or other disorder of fluid, electrolyte and acid-base balance.

In an embodiment, a decrease in the quantitative value of the ratio of omega-3 fatty acids to total fatty acids, when compared to the control sample or to the control value, may be indicative of the subject having an increased risk of developing the anemia and/or the metabolic condition related to the nutritional state and/or fluid balance of the blood, such as an iron deficiency anemia, other anemia, deficiency of B group vitamins, vitamin D deficiency, disorder of mineral metabolism, volume depletion and/or other disorder of fluid, electrolyte and acid-base balance.

In an embodiment, a decrease in the quantitative value of the ratio of omega-6 fatty acids to total fatty acids, when compared to the control sample or to the control value, may be indicative of the subject having an increased risk of developing the anemia and/or the metabolic condition related to the nutritional state and/or fluid balance of the blood, such as an iron deficiency anemia, other anemia, deficiency of B group vitamins, vitamin D deficiency, disorder of mineral metabolism, volume depletion and/or other disorder of fluid, electrolyte and acid-base balance.

In an embodiment, a decrease in the quantitative value of fatty acid degree of unsaturation, when compared to the control sample or to the control value, may be indicative of the subject having an increased risk of developing the anemia and/or the metabolic condition related to the nutritional state and/or fluid balance of the blood, such as an iron deficiency anemia, other anemia, deficiency of B group vitamins, vitamin D deficiency, disorder of mineral metabolism, volume depletion and/or other disorder of fluid, electrolyte and acid-base balance.

In an embodiment, a decrease in the quantitative value of docosahexaenoic acid, when compared to the control sample or to the control value, may be indicative of the subject having an increased risk of developing the anemia and/or the metabolic condition related to the nutritional state and/or fluid balance of the blood, such as an iron deficiency anemia, other anemia, deficiency of B group vitamins, vitamin D deficiency, disorder of mineral metabolism, volume depletion and/or other disorder of fluid, electrolyte and acid-base balance.

In an embodiment, a decrease in the quantitative value of linoleic acid, when compared to the control sample or to the control value, may be indicative of the subject having an increased risk of developing the anemia and/or the metabolic condition related to the nutritional state and/or fluid balance of the blood, such as an iron deficiency anemia, other anemia, deficiency of B group vitamins, vitamin D deficiency, disorder of mineral metabolism, volume depletion and/or other disorder of fluid, electrolyte and acid-base balance.

In an embodiment, a decrease in the quantitative value of omega-3 fatty acids, when compared to the control sample or to the control value, may be indicative of the subject having an increased risk of developing the anemia and/or the metabolic condition related to the nutritional state and/or fluid balance of the blood, such as an iron deficiency anemia, other anemia, deficiency of B group vitamins, vitamin D deficiency, disorder of mineral metabolism, volume depletion and/or other disorder of fluid, electrolyte and acid-base balance.

In an embodiment, a decrease in the quantitative value of omega-6 fatty acids, when compared to the control sample or to the control value, may be indicative of the subject having an increased risk of developing the anemia and/or the metabolic condition related to the nutritional state and/or fluid balance of the blood, such as an iron deficiency anemia, other anemia, deficiency of B group vitamins, vitamin D deficiency, disorder of mineral metabolism, volume depletion and/or other disorder of fluid, electrolyte and acid-base balance.

In an embodiment, an increase in the quantitative value of citrate, when compared to the control sample or to the control value, may be indicative of the subject having an increased risk of developing the anemia and/or the metabolic condition related to the nutritional state and/or fluid balance of the blood, such as an iron deficiency anemia, other anemia, deficiency of B group vitamins, vitamin D deficiency, disorder of mineral metabolism, volume depletion and/or other disorder of fluid, electrolyte and acid-base balance.

In an embodiment, an increase in the quantitative value of pyruvate, when compared to the control sample or to the control value, may be indicative of the subject having an increased risk of developing the anemia and/or the metabolic condition related to the nutritional state and/or fluid balance of the blood, such as an iron deficiency anemia, other anemia, deficiency of B group vitamins, vitamin D deficiency, disorder of mineral metabolism, volume depletion and/or other disorder of fluid, electrolyte and acid-base balance.

In an embodiment, an increase in the quantitative value of alanine, when compared to the control sample or to the control value, may be indicative of the subject having an increased risk of developing the anemia and/or the metabolic condition related to the nutritional state and/or fluid balance of the blood, such as an iron deficiency anemia, other anemia, deficiency of B group vitamins, vitamin D deficiency, disorder of mineral metabolism, volume depletion and/or other disorder of fluid, electrolyte and acid-base balance.

In an embodiment, a decrease in the quantitative value of glutamine, when compared to the control sample or to the control value, may be indicative of the subject having an increased risk of developing the anemia and/or the metabolic condition related to the nutritional state and/or fluid balance of the blood, such as an iron deficiency anemia, other anemia, deficiency of B group vitamins, vitamin D deficiency, disorder of mineral metabolism, volume depletion and/or other disorder of fluid, electrolyte and acid-base balance.

In an embodiment, a decrease in the quantitative value of histidine, when compared to the control sample or to the control value, may be indicative of the subject having an increased risk of the anemia and/or the metabolic condition related to the nutritional state and/or fluid balance of the blood, such as an iron deficiency anemia, other anemia, deficiency of B group vitamins, vitamin D deficiency, disorder of mineral metabolism, volume depletion and/or other disorder of fluid, electrolyte and acid-base balance.

In an embodiment, a decrease in the quantitative value of leucine, when compared to the control sample or to the control value, may be indicative of the subject having an increased risk of the anemia and/or the metabolic condition related to the nutritional state and/or fluid balance of the blood, such as an iron deficiency anemia, other anemia, deficiency of B group vitamins, vitamin D deficiency, disorder of mineral metabolism, volume depletion and/or other disorder of fluid, electrolyte and acid-base balance.

In an embodiment, an increase in the quantitative value of phenylalanine, when compared to the control sample or to the control value, may be indicative of the subject having an increased risk of developing the anemia and/or the metabolic condition related to the nutritional state and/or fluid balance of the blood, such as an iron deficiency anemia, other anemia, deficiency of B group vitamins, vitamin D deficiency, disorder of mineral metabolism, volume depletion and/or other disorder of fluid, electrolyte and acid-base balance.

In an embodiment, a decrease in the quantitative value of valine, when compared to the control sample or to the control value, may be indicative of the subject having an increased risk of developing the anemia and/or the metabolic condition related to the nutritional state and/or fluid balance of the blood, such as an iron deficiency anemia, other anemia, deficiency of B group vitamins, vitamin D deficiency, disorder of mineral metabolism, volume depletion and/or other disorder of fluid, electrolyte and acid-base balance.

In an embodiment, a risk score defined as β₀+β₁*concentration (glycoprotein acetyls)+β₂*concentration (albumin), where β₀ is an intercept term, β₁ is the weighted coefficient attributed to the concentration of glycoprotein acetyls, and β₂ is the weighted coefficient attributed to the concentration of albumin, may be indicative of the subject having an increased risk of developing the anemia and/or the metabolic condition related to the nutritional state and/or fluid balance of the blood, such as an iron deficiency anemia, other anemia, deficiency of B group vitamins, vitamin D deficiency, disorder of mineral metabolism, volume depletion and/or other disorder of fluid, electrolyte and acid-base balance.

In an embodiment, a risk score defined as β₀+β₁*concentration (glycoprotein acetyls)+β₂*concentration (fatty acid measure), where β₀ is an intercept term, β₁ is the weighted coefficient attributed to the concentration of glycoprotein acetyls, β₂ is the weighted coefficient attributed to the fatty acid measure, may be indicative of the subject having an increased risk of developing the anemia and/or the metabolic condition related to the nutritional state and/or fluid balance of the blood, such as an iron deficiency anemia, other anemia, deficiency of B group vitamins, vitamin D deficiency, disorder of mineral metabolism, volume depletion and/or other disorder of fluid, electrolyte and acid-base balance. The fatty acid measure may be one or more of the following fatty acids or their ratio to total fatty acids: docosahexaenoic acid, linoleic acid, omega-3 fatty acids, omega-6 fatty acids, monounsaturated fatty acids, and/or fatty acid degree of unsaturation.

In an embodiment, a risk score defined as β₀+β₁*concentration (glycoprotein acetyls)+β₂*concentration (albumin)+β₃*concentration (fatty acid measure), where β₀ is an intercept term, β₁ is the weighted coefficient attributed to the concentration of glycoprotein acetyls, β₂ is the weighted coefficient attributed to the concentration of albumin, and β₃ is the weighted coefficient attributed to the concentration of the fatty acid measure may be indicative of the subject having an increased risk of developing the anemia and/or the metabolic condition related to the nutritional state and/or fluid balance of the blood, such as an iron deficiency anemia, other anemia, deficiency of B group vitamins, vitamin D deficiency, disorder of mineral metabolism, volume depletion and/or other disorder of fluid, electrolyte and acid-base balance. The fatty acid measure may be one or more of the following fatty acids or their ratio to total fatty acids: docosahexaenoic acid, linoleic acid, omega-3 fatty acids, omega-6 fatty acids, monounsaturated fatty acids, and/or fatty acid degree of unsaturation.

The term “combination” may, at least in some embodiments, be understood such that the method comprises using a risk score, hazard ratio, odds ratio, and/or predicted absolute risk or relative risk calculated on the basis of the quantitative value(s) of the biomarkers. For example, if quantitative values of both glycoprotein acetyls and albumin are determined, the quantitative values of both biomarkers may be compared to the control sample or the control value separately, or a risk score, hazard ratio, odds ratio, and/or predicted absolute risk or relative risk calculated on the basis of the quantitative value(s) of both the biomarkers, and the risk score, hazard ratio, odds ratio, and/or predicted absolute risk or relative risk may be compared to the control sample or the control value.

In an embodiment, the method may comprise determining in the biological sample obtained from the subject a quantitative value of the following biomarkers:
glycoprotein acetyls;
albumin; and
comparing the quantitative value(s) of the biomarkers and/or a combination thereof to a control sample or to a control value(s);
wherein an increase or a decrease in the quantitative value(s) of the biomarkers and/or the combination thereof, when compared to the control sample or to the control value, is/are indicative of the subject having an increased risk of developing the anemia and/or the metabolic condition related to the nutritional state and/or fluid balance of the blood. An increase in the quantitative value of glycoprotein acetyls and a decrease in the quantitative value of albumin, when compared to the control sample or to the control value, may be indicative of the subject having an increased risk of developing the anemia and/or the metabolic condition related to the nutritional state and/or fluid balance of the blood.

In an embodiment, the method may comprise determining in the biological sample obtained from the subject a quantitative value of the following biomarkers:
glycoprotein acetyls,
at least one fatty acid measure(s) of the following fatty acids or their ratio to total fatty acids: docosahexaenoic acid, linoleic acid, omega-3 fatty acids, omega-6 fatty acids, monounsaturated fatty acids, and/or fatty acid degree of unsaturation; and
comparing the quantitative value(s) of the biomarkers and/or a combination thereof to a control sample or to a control value(s);
wherein an increase or a decrease in the quantitative value(s) of the biomarkers and/or the combination thereof, when compared to the control sample or to the control value, is/are indicative of the subject having an increased risk of developing the anemia and/or the metabolic condition related to the nutritional state and/or fluid balance of the blood. An increase in the quantitative value of glycoprotein acetyls and a decrease in the quantitative value of docosahexaenoic and/or linoleic acid and/or omega-3 fatty acids and/or omega-6 fatty acids and/or fatty acid degree of unsaturation and/or their ratio to total fatty acids, and/or an increase in the quantitative value of the ratio of monounsaturated fatty acid or oleic acid to total fatty acids, when compared to the control sample or to the control value, may be indicative of the subject having an increased risk of developing the anemia and/or the metabolic condition related to the nutritional state and/or fluid balance of the blood.

In an embodiment, the method may comprise determining in the biological sample obtained from the subject a quantitative value of the following biomarkers:
albumin,
at least one fatty acid measure(s) of the following fatty acids or their ratio to total fatty acids: docosahexaenoic acid, linoleic acid, omega-3 fatty acids, omega-6 fatty acids, monounsaturated fatty acids, and/or fatty acid degree of unsaturation; and
comparing the quantitative value(s) of the biomarkers and/or a combination thereof to a control sample or to a control value(s);
wherein an increase or a decrease in the quantitative value(s) of the biomarkers and/or the combination thereof, when compared to the control sample or to the control value, is/are indicative of the subject having an increased risk of developing the anemia and/or the metabolic condition related to the nutritional state and/or fluid balance of the blood. A decrease in the quantitative value of albumin and a decrease in the quantitative value of docosahexaenoic and/or linoleic acid and/or omega-3 fatty acids and/or omega-6 fatty acids and/or fatty acid degree of unsaturation and/or their ratio to total fatty acids, and/or an increase in the quantitative value of ratio of monounsaturated fatty acid or oleic acid to total fatty acids, when compared to the control sample or to the control value, may be indicative of the subject having an increased risk of developing the anemia or the metabolic condition related to the nutritional state and fluid balance of the blood.

In an embodiment, the method may comprise determining in the biological sample obtained from the subject a quantitative value of the following biomarkers:
glycoprotein acetyls,
albumin,
at least one fatty acid measure(s) of the following fatty acids or their ratio to total fatty acids: docosahexaenoic acid, linoleic acid, omega-3 fatty acids, omega-6 fatty acids, monounsaturated fatty acids, and/or fatty acid degree of unsaturation; and
comparing the quantitative value(s) of the biomarkers and/or a combination thereof to a control sample or to a control value(s);
wherein an increase or a decrease in the quantitative value(s) of the biomarkers and/or the combination thereof, when compared to the control sample or to the control value, is/are indicative of the subject having an increased risk of developing the anemia and/or the metabolic condition related to the nutritional state and/or fluid balance of the blood. An increase in the quantitative value of glycoprotein acetyls, a decrease in the quantitative value of albumin and a decrease in the quantitative value of docosahexaenoic and/or linoleic acid and/or omega-3 fatty acids and/or omega-6 fatty acids and/or fatty acid degree of unsaturation and/or their ratio to total fatty acids, and/or an increase in the quantitative value of ratio of monounsaturated fatty acid or oleic acid to total fatty acids, when compared to the control sample or to the control value, may be indicative of the subject having an increased risk of developing the anemia and/or the metabolic condition related to the nutritional state and/or fluid balance of the blood.

The following embodiments are also disclosed:
1. A method for determining whether a subject is at risk of developing an anemia and/or a metabolic condition related to the nutritional state and/or fluid balance of the blood;
wherein the method comprises determining in a biological sample obtained from the subject a quantitative value of at least one biomarker of the following in the biological sample:
   albumin,
   glycoprotein acetyls,
   ratio of docosahexaenoic acid to total fatty acids,
   ratio of linoleic acid to total fatty acids,
   ratio of monounsaturated fatty acids and/or of oleic acid to total fatty acids,
   ratio of omega-3 fatty acids to total fatty acids,
   ratio of omega-6 fatty acids to total fatty acids,
   fatty acid degree of unsaturation,
   docosahexaenoic acid,
   linoleic acid,
   omega-3 fatty acids,
   omega-6 fatty acids,
   citrate,
   pyruvate,
   alanine,
   glutamine,
   histidine,
   leucine,
   phenylalanine,
   valine; and
comparing the quantitative value(s) of the at least one biomarker to a control sample or to a control value;
wherein an increase or a decrease in the quantitative value(s) of the at least one biomarker, when compared to the control sample or to the control value, is/are indicative of the subject having an increased risk of developing the anemia and/or the metabolic condition related to the nutritional state and/or fluid balance of the blood.
2. The method according to embodiment 1, wherein the method comprises determining in the biological sample quantitative values of a plurality of the biomarkers, such as two, three, four, five or more of the biomarkers.
3. The method according to any one of embodiments 1-2, wherein the at least one biomarker comprises or is glycoprotein acetyls.
4. The method according to any one of embodiments 1-3, wherein the method comprises determining in the biological sample obtained from the subject a quantitative value of the following biomarkers:
   glycoprotein acetyls;
   albumin; and
comparing the quantitative value(s) of the biomarkers to a control sample or to a control value(s);
wherein an increase or a decrease in the quantitative value(s) of the biomarkers, when compared to the control sample or to the control value, is/are indicative of the subject having an increased risk of developing the anemia and/or the metabolic condition related to the nutritional state and/or fluid balance of the blood.
5. The method according to any one of embodiments 1-4, wherein the method comprises determining in the biological sample obtained from the subject a quantitative value of the following biomarkers:
   glycoprotein acetyls,
   at least one fatty acid measure(s) of the following: ratio of docosahexaenoic acid to total fatty acids, docosahexaenoic acid, ratio of linoleic acid to total fatty acids, linoleic acid, ratio of monounsaturated fatty acids and/or of oleic acid to total fatty acids, ratio of omega-3 fatty acids to total fatty acids, omega-3 fatty acids, ratio of omega-6 fatty acids to total fatty acids, omega-6 fatty acids, fatty acid degree of unsaturation; and
comparing the quantitative value(s) of the biomarkers to a control sample or to a control value(s);
wherein an increase or a decrease in the quantitative value(s) of the biomarkers, when compared to the control sample or to the control value, is/are indicative of the subject having an increased risk of developing the anemia and/or the metabolic condition related to the nutritional state and/or fluid balance of the blood.
6. The method according to any one of embodiments 1-5, wherein the anemia and/or the metabolic condition related to the nutritional state and/or fluid balance of the blood comprises or is iron deficiency anemia (D50); vitamin B12 deficiency anemia (D51); other aplastic anemia or other bone marrow failure syndrome (D61); anemia in chronic diseases classified elsewhere (D63); other anemia (D64); deficiency of other B group vitamins (E53); vitamin D deficiency (E55); deficiency of other nutrient elements (E61); disorder of mineral metabolism (E83); volume depletion (E86); or other disorder of fluid, electrolyte and acid-base balance (E87).
7. The method according to any one of embodiments 1-6, wherein the anemia and/or the metabolic condition related to the nutritional state and/or fluid balance of the blood comprises or is other iron deficiency anemia (D50.8); iron deficiency anemia, unspecified (D50.9); vitamin B12 deficiency anemia due to intrinsic factor deficiency (D51.0); aplastic anemia, unspecified (D61.9); anemia in neoplastic disease (D63.0); disorder of phosphorus metabolism and/or phosphatases (E83.3); anemia, unspecified (D64.9); deficiency of other specified B group vitamins (E53.8; vitamin D deficiency, unspecified (E55.9); iron deficiency (E61.1); disorder of magnesium metabolism (E83.4); disorder of calcium metabolism (E83.5); hyperosmolality and/or hypernatremia (E87.0); hypo-osmolality and/or hyponatremia (E87.1); acidosis (E87.2); hyperkalemia (E87.5); and/or hypokalemia (E87.6).
8. The method according to any one of embodiments 1-7, wherein the anemia and/or the metabolic condition related to the nutritional state and/or fluid balance of the blood comprises or is iron deficiency anemia, other anemia, vitamin deficiency, disorder of mineral metabolism, volume depletion, or other disorder of fluid, electrolyte and/or acid-base balance.
9. The method according to any one of embodiments 1-8, wherein the quantitative value of the at least one biomarker is/are measured using nuclear magnetic resonance spectroscopy.
10. The method according to any one of embodiments 1-9, wherein the method further comprises determining whether the subject is at risk of developing the anemia and/or the metabolic condition related to the nutritional state and/or fluid balance of the blood using a risk score, hazard ratio, odds ratio, and/or predicted absolute risk or relative risk calculated on the basis of the quantitative value(s) of the at least one biomarker or of the plurality of the biomarkers.

### EXAMPLES

Reference will now be made in detail to various embodiments, an example of which is illustrated in the accompanying drawings. The description below discloses some embodiments in such a detail that a person skilled in the art is able to utilize the embodiments based on the disclosure. Not all steps or features of the embodiments are discussed in detail, as many of the steps or features will be obvious for the person skilled in the art based on this specification.

### Abbreviations Used in the Figures

DHA %: Ratio of docosahexaenoic acid to total fatty acids
LA %: Ratio of linoleic acid to total fatty acids
Omega-3%: Ratio of omega-3 fatty acids to total fatty acids
Omega-6%: Ratio of omega-6 fatty acids to total fatty acids
MUFA %: Ratio of monounsaturated fatty acids to total fatty acids
DHA: Docosahexaenoic acid
LA: Linoleic acid
MUFA: Monounsaturated fatty acids
Omega-3: Omega-3 fatty acids
Omega-6: Omega-6 fatty acids
Unsaturation: Fatty acid degree of unsaturation
CI: confidence interval
SD: standard deviation
BMI: Body mass index

### Example 1

Biomarker measures quantified by nuclear magnetic resonance (NMR) were investigated as to whether they could be predictive of an anemia and/or a metabolic condition related to the nutritional state and/or fluid balance of the blood, such as an iron deficiency anemia, other anemias, deficiency of B group vitamins, vitamin D deficiency, disorder of mineral metabolism, volume depletion and/or other disorder of fluid, electrolyte and acid-base balance. All analyses were conducted based on the UK Biobank, with approximately 115,000 study participants with blood biomarker data from NMR spectroscopy available.

Study Population

Details of the design of the UK Biobank have been reported by Sudlow et al 2015, PLoS Med. 2015; 12(3):e1001779. Briefly, UK Biobank recruited 502 639 participants aged 37-73 years in 22 assessment centres across the UK. All participants provided written informed consent and ethical approval was obtained from the North West Multi-Center Research Ethics Committee. Blood samples were drawn at baseline between 2007 and 2010. No selection criteria were applied to the sampling.

Biomarker Profiling

From the entire UK Biobank population, a random subset of baseline plasma samples from 118 466 individuals were measured using the Nightingale NMR biomarker platform (Nightingale Health Ltd, Finland). This blood analysis method provides simultaneous quantification of many blood biomarkers, including lipoprotein lipids, circulating fatty acids, and various low-molecular weight metabolites including amino acids, ketone bodies and gluconeogenesis-related metabolites in molar concentration units. Technical details and epidemiological applications have been reviewed (Soininen et al 2015, Circ Cardiovasc Genet; 2015; 8:192-206; Würtz et al 2017, Am J Epidemiol 2017; 186:1084-1096). Values outside four interquartile ranges from median were considered as outliers and excluded.

Epidemiological Analyses of Biomarker Relations with Risk of an Anemia and/or a Metabolic Condition Related to the Nutritional State and/or Fluid Balance of the Blood

The blood biomarker associations with the risk for an anemia and/or a metabolic condition related to the nutritional state and/or fluid balance of the blood were conducted based on UK Biobank data. Analyses focused on the relation of biomarkers to the occurrence of an anemia and/or a metabolic condition related to the nutritional state and/or fluid balance of the blood after the blood samples were collected, to determine if the individual biomarkers associate with the risk for future development of an anemia and/or a metabolic condition related to the nutritional state and/or fluid balance of the blood. Examples using multi-biomarker scores, in the form weighted sums of biomarkers, were also explored to see if they could be predictive even more strongly than each individual biomarker.

Information on the disease events occurring after the blood samplings for all study participants were recorded from UK Hospital Episode Statistics data and death registries. All analyses are based on first occurrence of diagnosis, so that individuals with recorded diagnosis of the given disease prior to blood sampling were omitted from the statistical analyses. A composite endpoint of Any Anemia and/or Any Metabolic Condition Related to the Nutritional State and/or Fluid Balance of the Blood was defined based on any incident occurrence of ICD-10 diagnoses D50-D89, E50-E64 or E70-E88, except E78 (disorders of lipoprotein metabolism). More refined subtypes of the anemias and/or metabolic conditions related to the nutritional state and/or fluid balance of the blood were defined according to the ICD-10 diagnoses listed in Table 1.

The registry-based follow-up was from blood sampling in 2007-2010 through to 2020 (approximately 1 100 000 person-years). Specific diseases which had <150 disease events recorded during follow-up were left out of scope.

For biomarker association testing, Cox proportional-hazard regression models adjusted for age, sex, and UK Biobank assessment centre were used. Results were plotted in magnitudes per standard deviation of each biomarker measure to allow direct comparison of association magnitudes.

Summary of Results

Baseline characteristics of the study population for biomarker analyses vs future risk of an anemia and/or a metabolic condition related to the nutritional state and/or fluid balance of the blood are shown in Table 1. The number of incident disease events occurring after the blood sampling is listed for all the conditions analysed.

TABLE 1
Clinical characteristics of study participants
and the number of incident disease events analysed.

| | |
|---|---|
| Total number of individuals with blood | 118 456 |
| samples analysed | |
| Study setting | Population |
| | sample of |
| | study |
| | volunteers |
| | from the UK |
| Percentage of women | 54.1% |
| Age range (years) | 39-71 |
| Median age (years) | 58 |
| Median BMI (kg/m2) | 26.8 |
| Follow-up time for disease events after | 10-14 years |
| blood sampling | |
| Diseases with similar biomarker rela- | Number of in- |
| tions | dividuals who |
| | developed the |
| | specified |
| | disease after |
| | the blood |
| | sampling |
| Any Anemia and/or Any Metabolic Condi- | 12276 |
| tion Related to the Nutritional State | |
| and/or Fluid Balance of the Blood: any | |
| occurrence of D50-D89, E50-E64 or E70- | |
| E88, except E78, ICD-10 codes | |
| Specific Anemia and/or Metabolic Condi- | |
| tion Related to the Nutritional State | |
| and/or Fluid Balance of the Blood (de- | |
| fined by 3-character ICD-10 codes) | |
| D50: Iron deficiency anemia | 3714 |
| D51: Vitamin B12 deficiency anemia | 339 |
| D61: Other aplastic anemias and other | 228 |
| bone marrow failure syndromes | |
| D63: Anemia in chronic diseases classi- | 292 |
| fied elsewhere | |
| D64: Other anemias | 4485 |
| E53: Deficiency of other B group vita- | 566 |
| mins | |
| E55: Vitamin D deficiency | 900 |
| E61: Deficiency of other nutrient ele- | 177 |
| ments | |
| E83: Disorders of mineral metabolism | 1313 |
| E86: Volume depletion | 1802 |
| E87: Other disorders of fluid, electro- | 3752 |
| lyte and acid-base balance | |
| Specific Anemia and/or Metabolic Condi- | |
| tion Related to the Nutritional State | |
| and/or Fluid Balance of the Blood (de- | |
| fined by 4-character ICD-10 codes) | |
| D50.8: Other iron deficiency anemias | 1229 |
| D50.9: Iron deficiency anemia, unspeci- | 2859 |
| fied | |
| D51.0: Vitamin B12 deficiency anemia | 278 |
| due to intrinsic factor deficiency | |
| D61.9: Aplastic anemia, unspecified | 208 |
| D63.0: Anemia in neoplastic disease | 157 |
| D64.9: Anemia, unspecified | 4472 |
| E53.8: Deficiency of other specified B | 544 |
| group vitamins | |
| E55.9: Vitamin D deficiency, unspeci- | 900 |
| fied | |
| E61.1: Iron deficiency | 157 |
| E83.3: Disorders of phosphorus metabo- | 235 |
| lism and phosphatases | |
| E83.4: Disorders of magnesium metabo- | 413 |
| lism | |
| E83.5: Disorders of calcium metabolism | 654 |
| E87.0: Hyperosmolality and hyper- | 228 |
| natremia | |
| E87.1: Hypo-osmolality and hyponatremia | 1434 |
| E87.2: Acidosis | 791 |
| E87.5: Hyperkalemia | 646 |
| E87.6: Hypokalemia | 1105 |
| E87.7: Fluid overload | 462 |

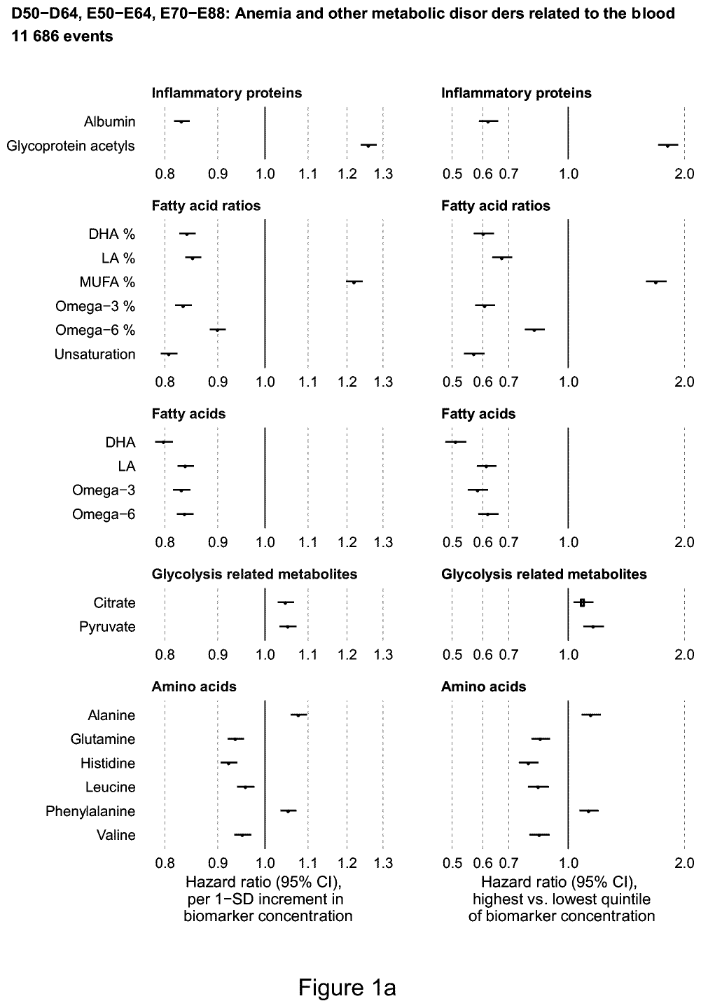

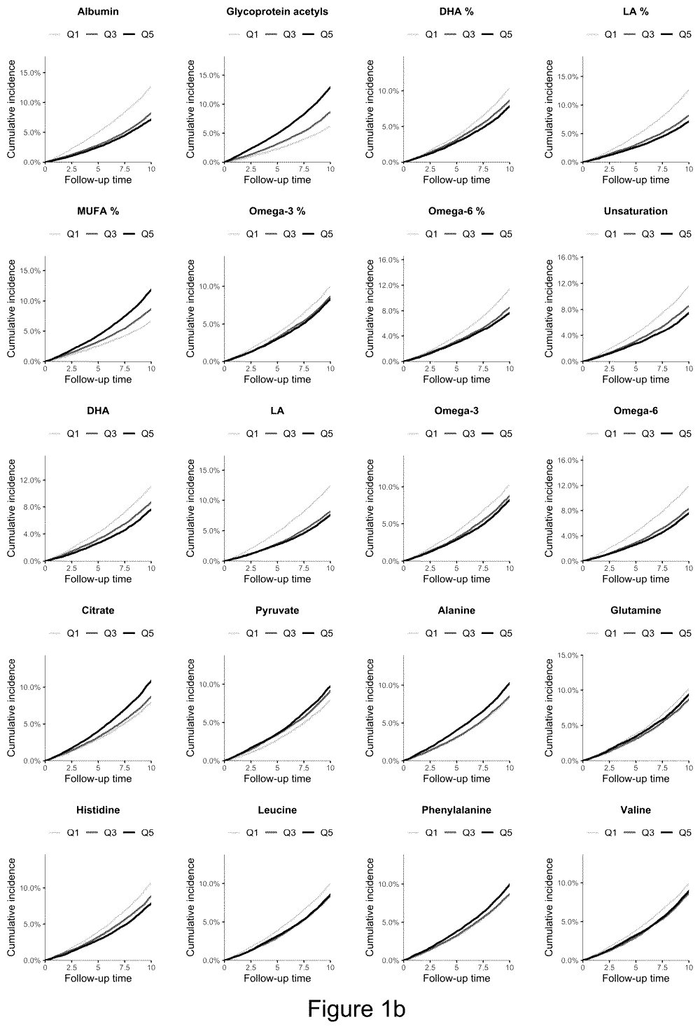

FIG. 1a shows the hazard ratios for the 20 blood biomarkers with the future risk of Any Anemia and/or Any Metabolic Condition Related to the Nutritional State and/or Fluid Balance of the Blood (ICD-10 codes D50-D89, E50-E64 and E70-E88, except E78). The left-hand side of the figure shows the hazard ratios when the biomarkers are analysed in absolute concentrations, scaled to standard deviations of the study population. The right-hand side shows the corresponding hazard ratios when individuals in the highest quintile of the biomarker concentration are compared to those in the lowest quintile. The results are based on statistical analyses of over 115,000 individuals from the UK Biobank, out of whom 12,276 developed an anemia and/or a metabolic condition related to the nutritional state and/or fluid balance of the blood (defined as diagnosis D50-D89, E50-E64 or E70-E88 (except E78) in the hospital registries, or in the death records) during approximately 10 years of follow-up. The analyses were adjusted for age, sex, and UK Biobank assessment centre in Cox proportional-hazard regression models. P-values were P<0.0001 (corresponding to multiple testing correction) for all associations. These results demonstrate that the 20 individual biomarkers are predictive of the risk for anemia or other metabolic conditions related to the blood in general population settings.

FIG. 1b shows the Kaplan-Meier plots of the cumulative risk for an anemia and/or a metabolic condition related to the nutritional state and/or fluid balance of the blood for each of the 20 blood biomarkers according to the lowest, middle, and highest quintiles of biomarker concentrations. The results are based on statistical analyses of over 115,000 individuals from the UK Biobank, out of whom 12,276 developed an anemia and/or a metabolic condition related to the nutritional state and/or fluid balance of the blood. These results further demonstrate that the 20 individual biomarkers are predictive of the risk for an anemia and/or a metabolic condition related to the nutritional state and/or fluid balance of the blood in general population settings.

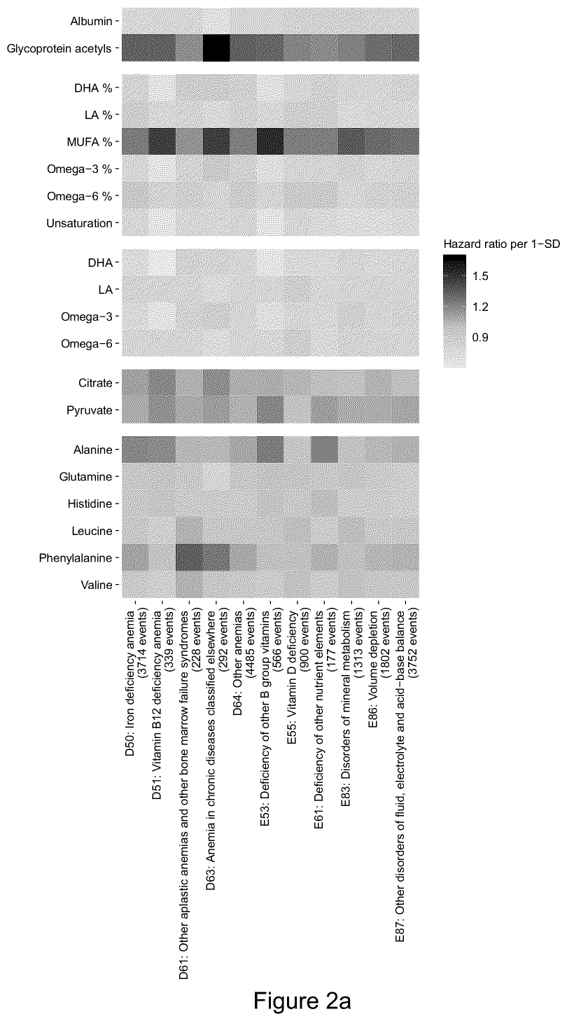

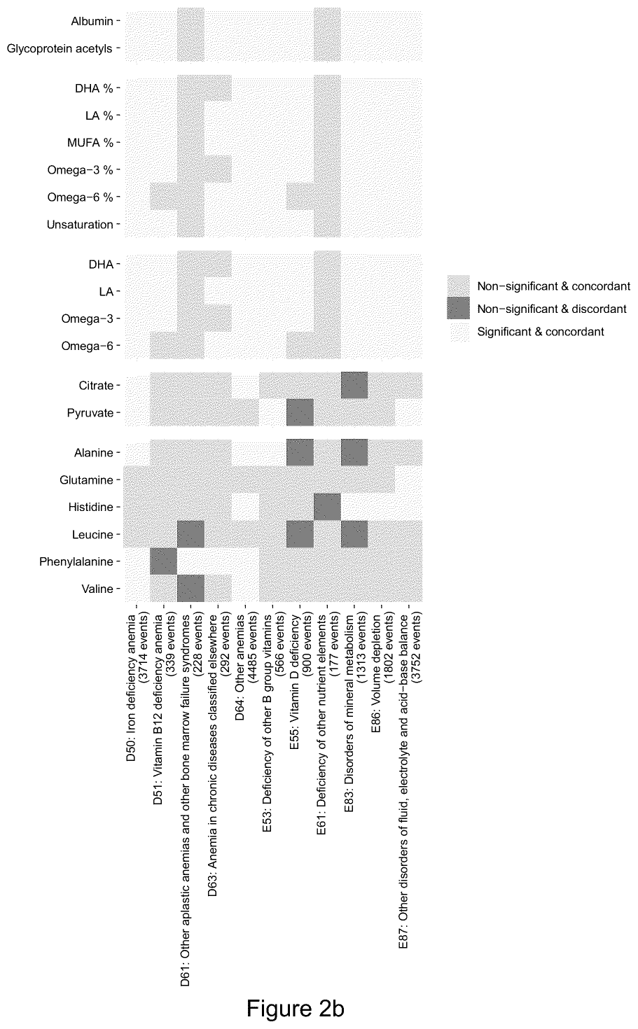

FIG. 2a shows the hazard ratios for 20 blood biomarkers for future onset of 11 specific anemias and/or metabolic conditions related to the nutritional state and/or fluid balance of the blood, defined by 3-character ICD-10 diagnosis codes. The results illustrate that the pattern of biomarker associations is highly consistent for the 11 different specific disorders.

FIG. 2b shows the consistency of all the biomarker associations with the 11 specific anemias and/or metabolic conditions related to the nutritional state and/or fluid balance of the blood (defined by 3-character ICD-10 diagnosis codes) compared to the “Any Anemia and/or Any Metabolic Condition Related to the Nutritional State and/or Fluid Balance of the Blood” definition. The biomarker associations were all in the same direction of association as for “Any Anemia and/or Any Metabolic Condition Related to the Nutritional State and/or Fluid Balance of the Blood” or not statistically significant in the discordant direction. Any biomarker combination that strongly predicts “Any Anemia and/or Any Metabolic Condition Related to the Nutritional State and/or Fluid Balance of the Blood” will therefore also be predictive of all the listed specific anemias and/or metabolic conditions related to the nutritional state and/or fluid balance of the blood.

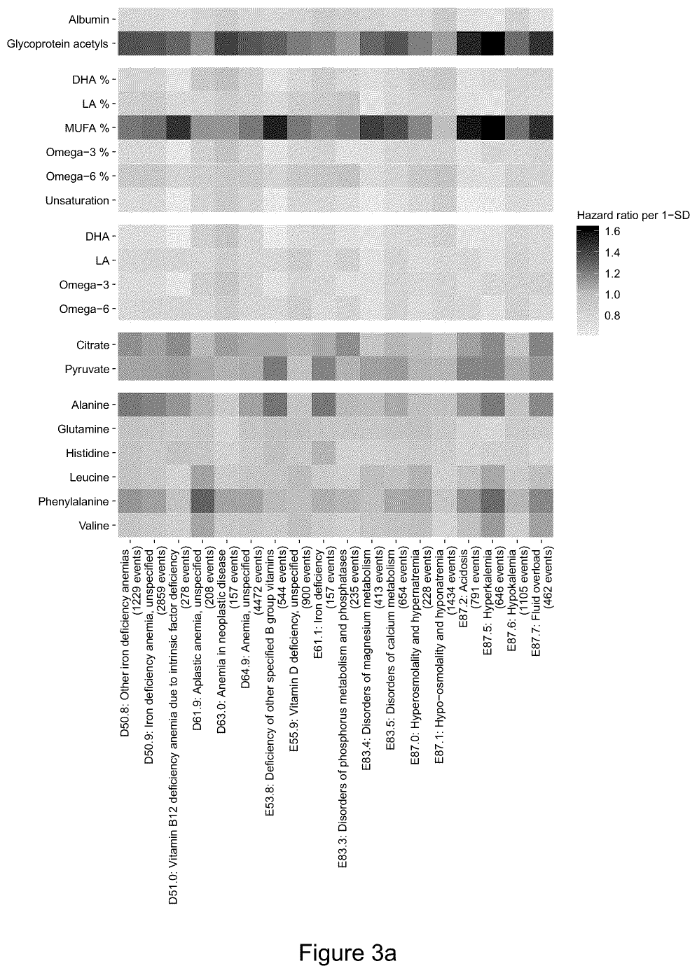

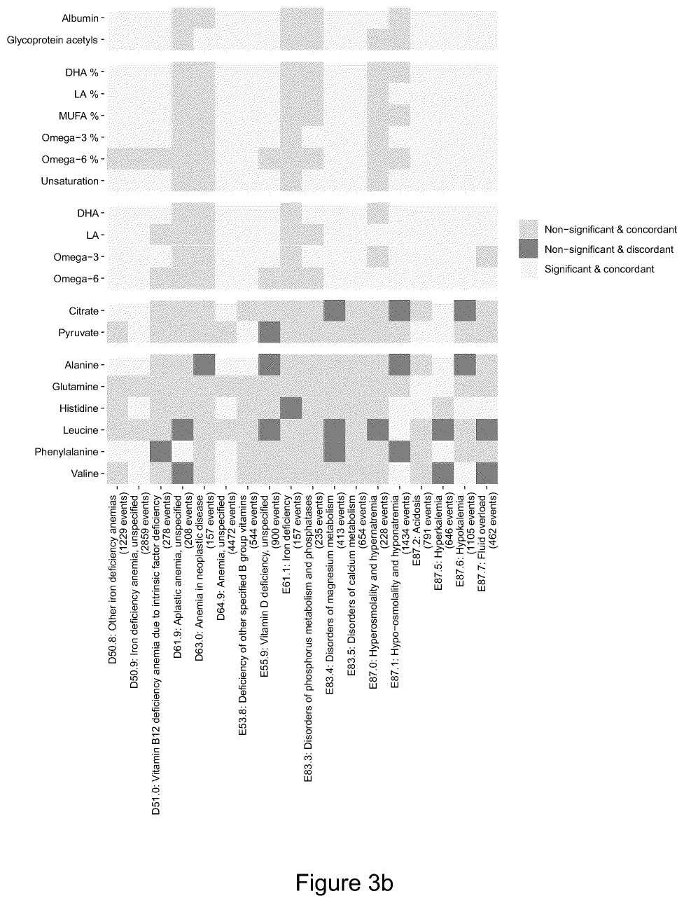

FIG. 3a shows the hazard ratios for 20 blood biomarkers for future onset of 18 specific types of anemias and/or metabolic conditions related to the nutritional state and/or fluid balance of the blood, defined by 4-character ICD-10 diagnosis codes. The results illustrate that the pattern of biomarker associations is highly consistent for all the 18 specific disorders.

FIG. 3b shows the consistency of all the biomarker associations with the 18 specific anemias and/or metabolic conditions related to the nutritional state and/or fluid balance of the blood (defined by 4-character ICD-10 diagnosis codes) compared to the “Any Anemia and/or Any Metabolic Condition Related to the Nutritional State and/or Fluid Balance of the Blood” definition. Generally, the biomarker associations are all in the same direction of association as for “Any Anemia and/or Any Metabolic Condition Related to the Nutritional State and/or Fluid Balance of the Blood” or not statistically significant in the discordant direction. Any biomarker combination that strongly predicts “Any Anemia and/or Any Metabolic Condition Related to the Nutritional State and/or Fluid Balance of the Blood” will therefore also be predictive of all the listed specific anemias and/or metabolic conditions related to the nutritional state and/or fluid balance of the blood.

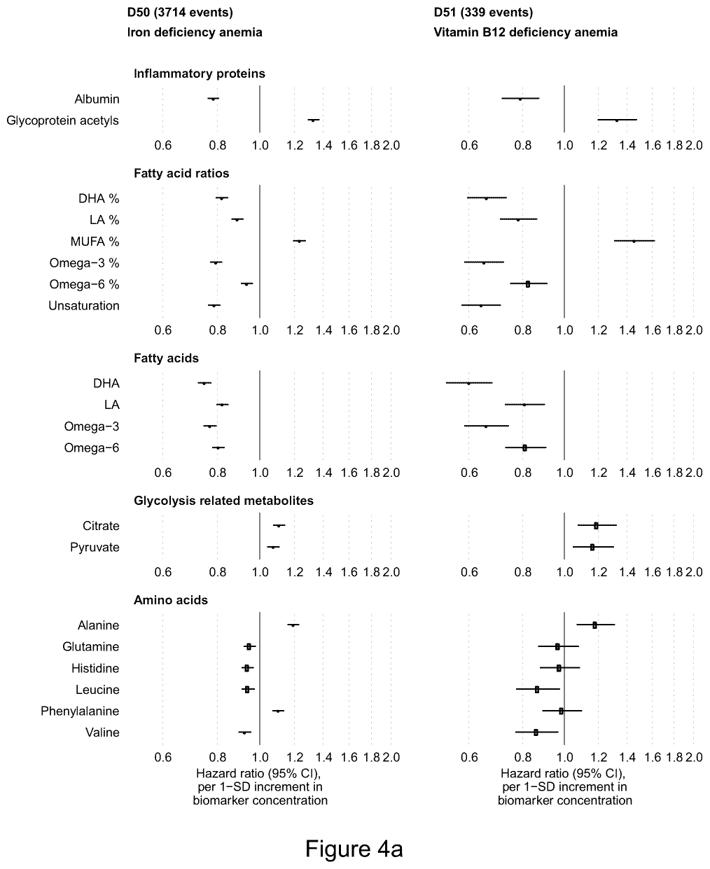

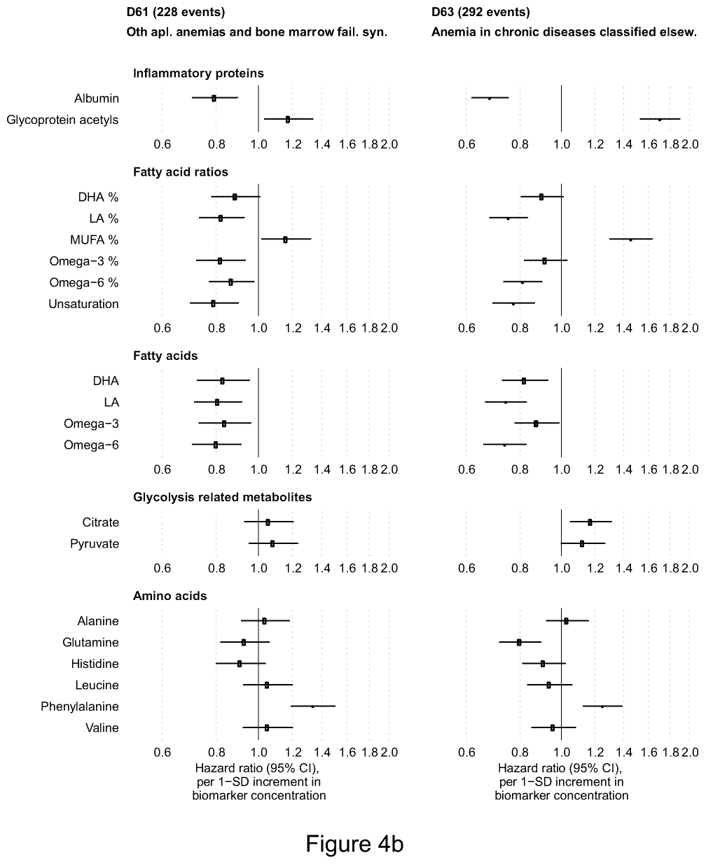

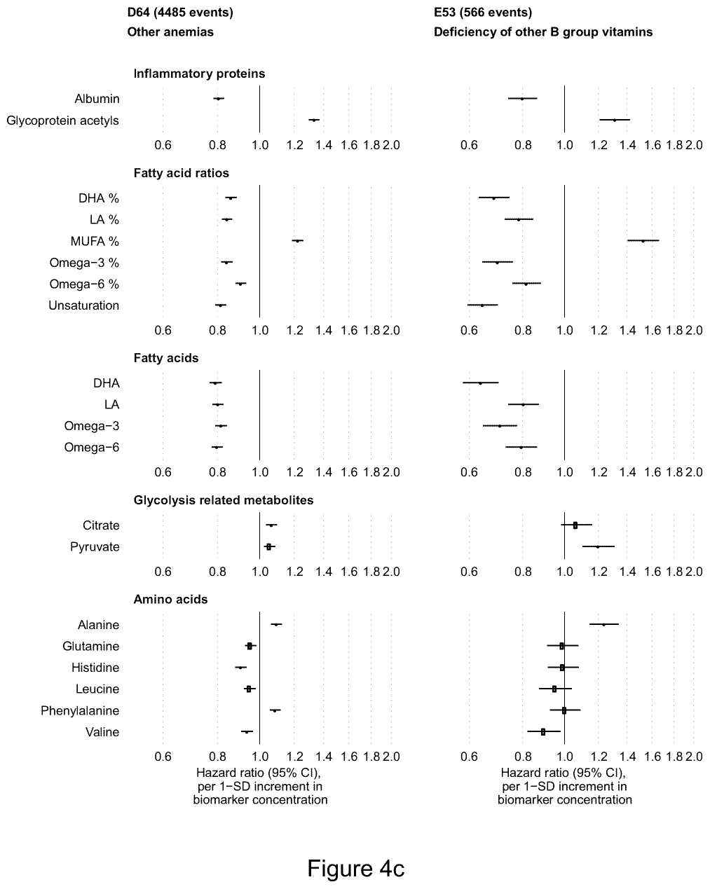

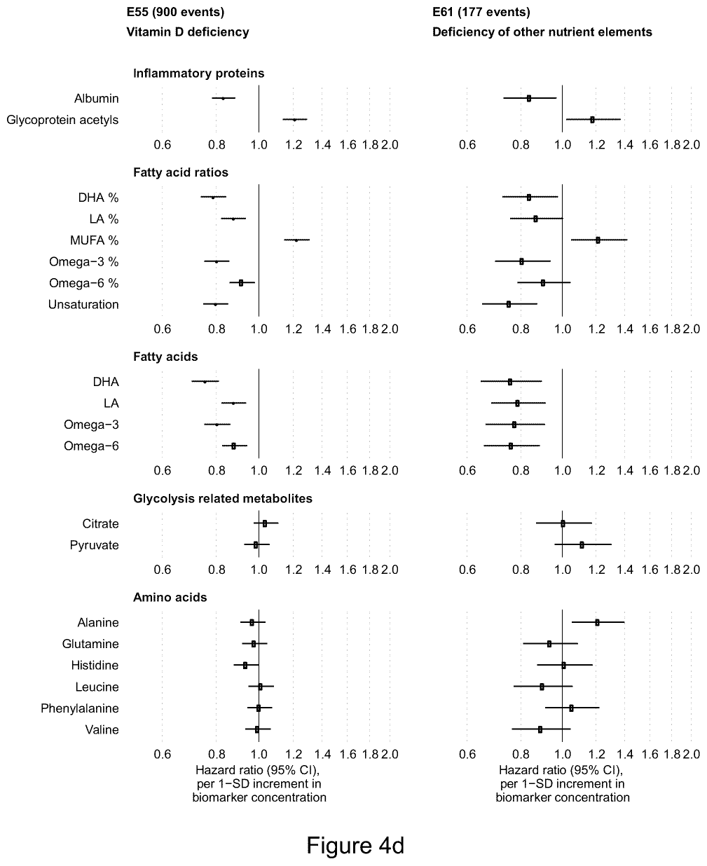

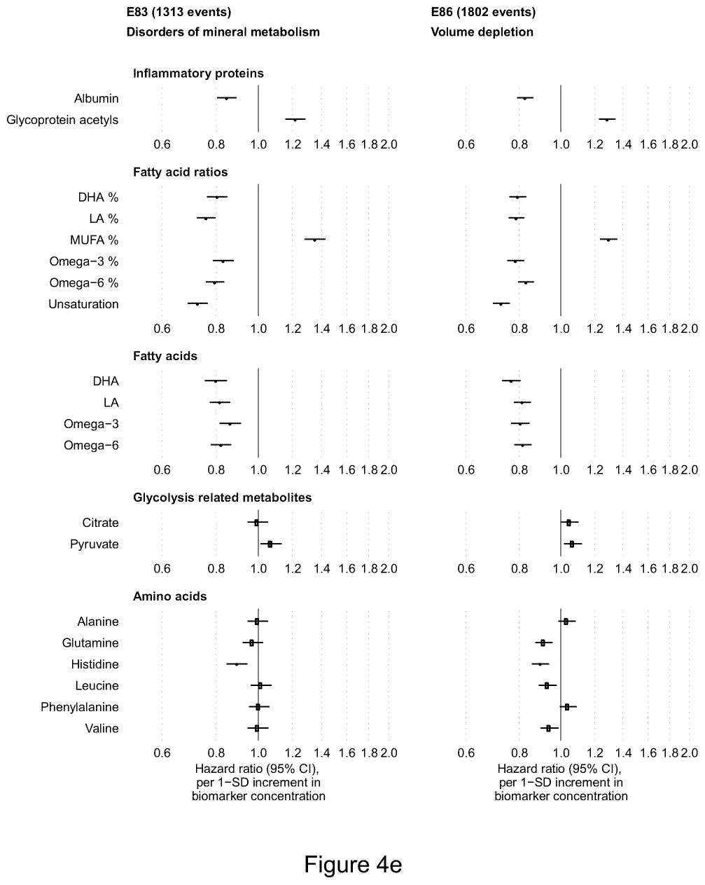

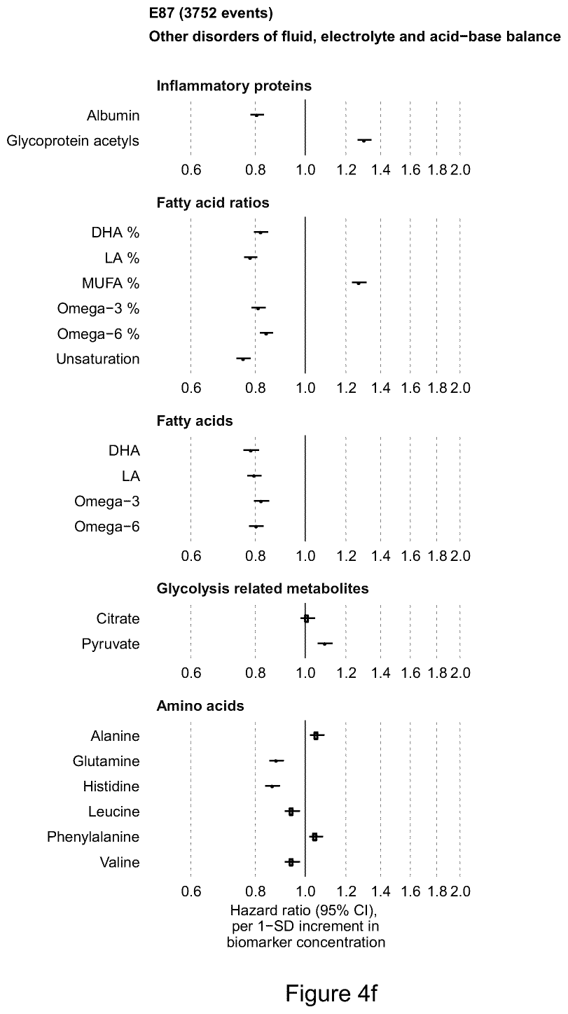

FIGS. 4a-f show the hazard ratios for 20 blood biomarkers with future onset of each of the 11 specific anemias and/or metabolic conditions related to the nutritional state and/or fluid balance of the blood (defined by 3-character ICD diagnosis codes) studied here. The hazard ratios are shown in absolute concentrations, scaled to the standard deviation of each biomarker. The results are based on statistical analyses of over 115,000 individuals from the UK Biobank; the number of individuals who developed the specific disease during approximately 10 years of follow-up is indicated on the top of each plot. Filled circles denote that the P-value for association was P<0.0001 (corresponding to multiple testing correction), and open circles denote that the P-value for association was P>0.0001. The analyses were adjusted for age, sex, and UK Biobank assessment centre using Cox proportional-hazard regression models.

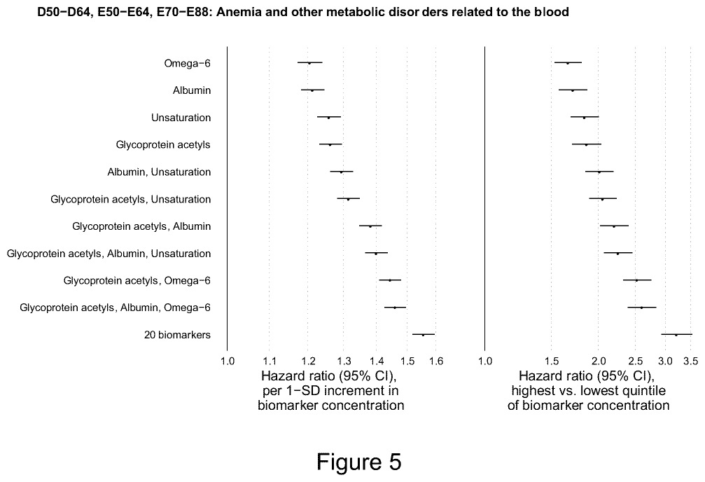

FIG. 5 shows examples of stronger associations with Any Anemia and/or Any Metabolic Condition Related to the Nutritional State and/or Fluid Balance of the Blood when two or more biomarkers are combined. The hazard ratios with future risk of Any Anemia and/or Any Metabolic Condition Related to the Nutritional State and/or Fluid Balance of the Blood (composite endpoint of ICD-10 codes D50-D89, E50-E64 and E70-E88, except E78) are shown for selected combinations of pairs of biomarkers, and examples of biomarker scores. The results were similar with many other combinations, in particular inclusion of different fatty acid measures in addition to albumin and glycoprotein acetyls. The biomarker scores are combined in the form of Σᵢ [symbol] βᵢ*cᵢ [symbol] +β₀; where i is the index of summation over individual biomarkers, βᵢ is the weighted coefficient attributed to biomarker i, cᵢ is the blood concentration of biomarker i and β₀ is an intercept term. βᵢ multipliers are defined according to the multivariate association magnitude with the risk for Any Anemia and/or Any Metabolic Condition Related to the Nutritional State and/or Fluid Balance of the Blood, examined in the statistical analyses of the UK Biobank study for the respective combination of biomarkers. The enhancements in association magnitudes were similar for the 11 specific types of anemias and/or metabolic conditions related to the nutritional state and/or fluid balance of the blood listed in Table 1 as those shown here for Any Anemia and/or Any Metabolic Condition Related to the Nutritional State and/or Fluid Balance of the Blood.

### Illustrations of Intended Use: Biomarker Scores for Risk Prediction of Anemia or Other Metabolic Conditions Related to the Blood.

For illustration of intended applications related to prediction of an anemia and/or a metabolic condition related to the nutritional state and/or fluid balance of the blood, further epidemiological analyses are illustrated below. These applications are exemplified for prediction of the risk for an iron deficiency anemia, other anemias, deficiency of B group vitamins, vitamin D deficiency, disorder of mineral metabolism, volume depletion and/or other disorder of fluid, electrolyte and acid-base balance. Similar results apply to the other anemias and/or metabolic conditions related to the nutritional state and/or fluid balance of the blood listed in Table 1. Results are shown for a biomarker score combining the 20 biomarkers featured in FIGS. 1-6. Similar results, albeit slightly weaker, are obtained with combinations of only two or three individual biomarkers.

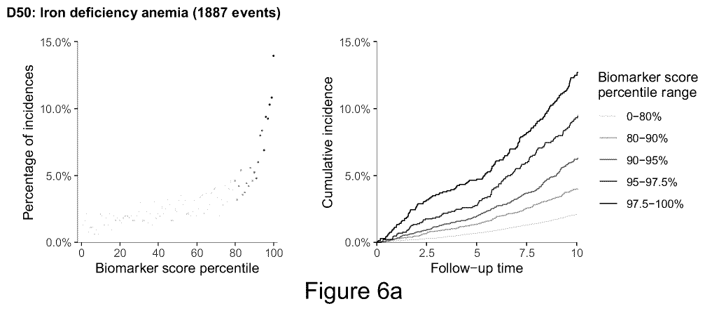

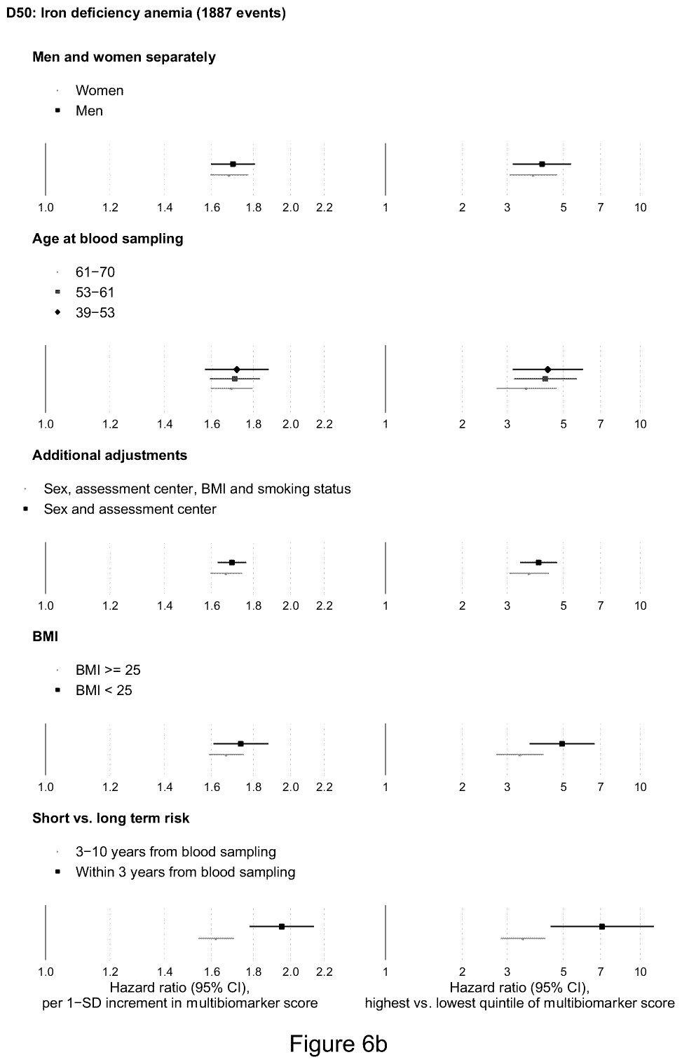

FIG. 6a shows the increase in risk for iron deficiency anemia (ICD-10 code D50) along with increasing levels of a biomarker risk score composed of the weighted sum of 20 biomarkers. On the left-hand side, the risk increase is plotted in the form of gradient percentile plots, showing the proportion of individuals who developed iron deficiency anemia during follow-up when binning individuals into the percentiles of the biomarker levels. Each dot corresponds to approximately 500 individuals. The Kaplan-Meier plots on the right-hand side illustrate the cumulative risk for iron deficiency anemia during follow-up for selected quantiles of the plurality-biomarker-score. Both plots serve to demonstrate that the risk is increasing non-linearly in the high end of the distribution of the plurality biomarker score. The plots are shown for the validation set part of the study population, i.e. 50% which was not included for derivation of the plurality-biomarker-scores (n=58 594 individuals).

FIG. 6b shows the hazard ratio of the same plurality-biomarker score with future onset of iron deficiency anemia (ICD-code D50) when accounting for relevant risk factor characteristics of the study participants. The first two panels of results demonstrate that the risk prediction applications are consistent for men and women, as well as for people at different ages at time of blood sampling. The third panel shows that the magnitude of the hazard ratio is only modestly attenuated when accounting for body mass index and smoking status in the statistical modelling. The fourth panel shows that the predictive associations are strong both for lean individuals (BMI<25) as well as overweight or obese individuals (BMI>25). The last panel shows that the hazard ratio is substantially stronger when focusing on short-term risk of iron deficiency anemia, here illustrated by using diagnoses recorded during the first 3 years after the blood samples were taken.

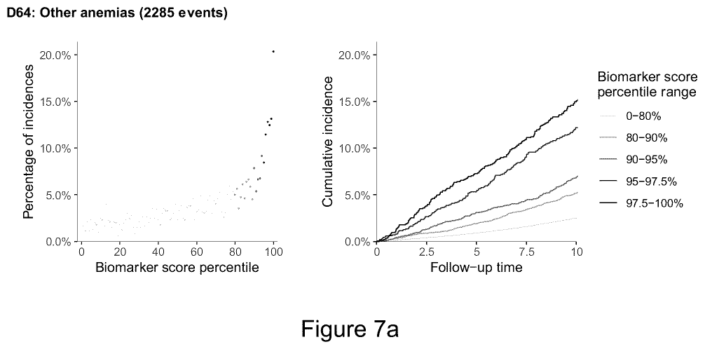

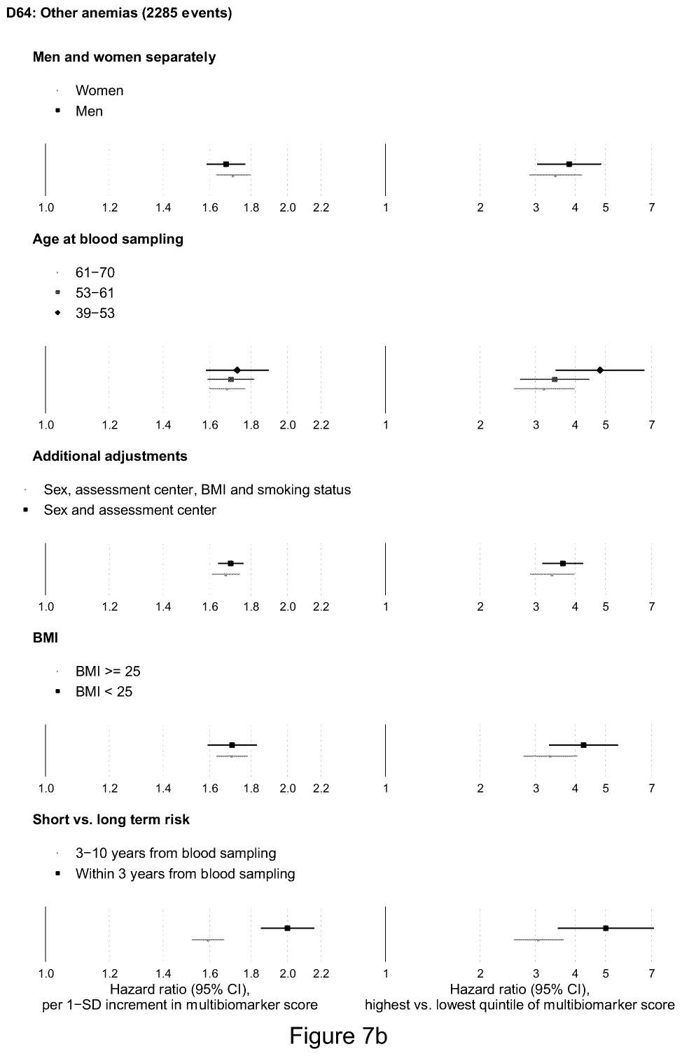

FIG. 7a shows the increase in risk for other anemias (ICD-10 code D64) along with increasing levels of a biomarker risk score composed of the weighted sum of 20 biomarkers. On the left-hand side, the risk increase is plotted in the form of gradient percentile plots, showing the proportion of individuals who developed other anemias during follow-up when binning individuals into the percentiles of the biomarker levels. Each dot corresponds to approximately 500 individuals. The Kaplan-Meier plots on the right-hand side illustrate the cumulative risk for other anemias during follow-up for selected quantiles of the plurality-biomarker-score. Both plots serve to demonstrate that the risk is increasing non-linearly in the high end of the distribution of the plurality biomarker score. The plots are shown for the validation set part of the study population, i.e. 50% which was not included for derivation of the plurality-biomarker-scores (n=58 594 individuals).

FIG. 7b shows the hazard ratio of the same plurality-biomarker score with future onset of other anemias (ICD-10 code D64) when accounting for relevant risk factor characteristics of the study participants. The first two panels of results demonstrate that the risk prediction applications are consistent for men and women, as well as for people at different ages at time of blood sampling. The third panel shows that the magnitude of the hazard ratio is only modestly attenuated when accounting for body mass index and smoking status in the statistical modelling. The fourth panel shows that the predictive associations are strong both for lean individuals (BMI<25) as well as overweight or obese individuals (BMI>25). The last panel shows that the hazard ratio is substantially stronger when focusing on short-term risk of other anemias, here illustrated by using diagnoses recorded during the first 3 years after the blood samples were taken.

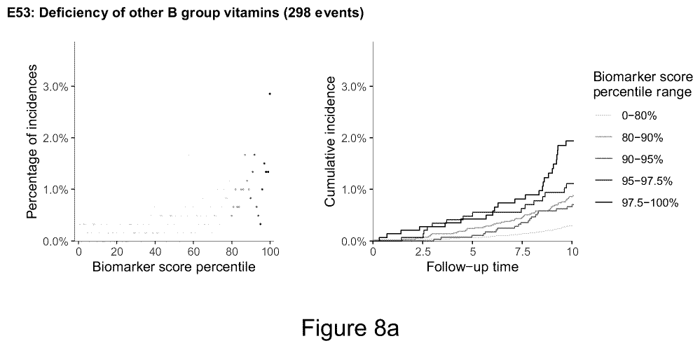

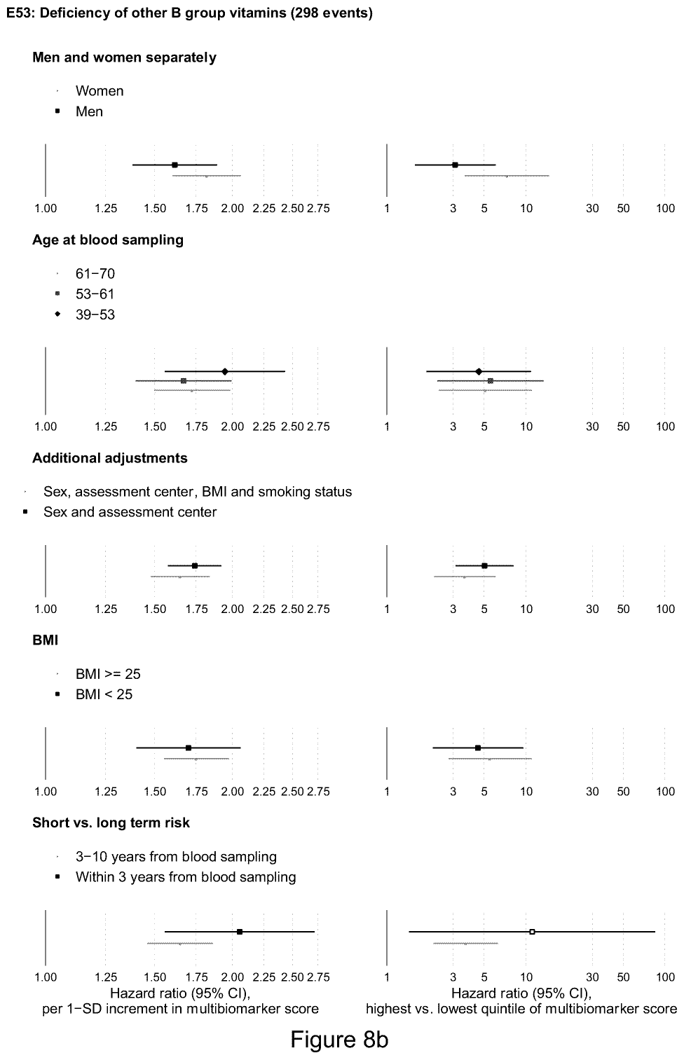

FIG. 8a shows the increase in risk for deficiency of other B group vitamins (ICD-10 code E53) along with increasing levels of a biomarker risk score composed of the weighted sum of biomarkers. On the left-hand side, the risk increase is plotted in the form of gradient percentile plots, showing the proportion of individuals who developed deficiency of other B group vitamins during follow-up when binning individuals into the percentiles of the biomarker levels. Each dot corresponds to approximately 500 individuals. The Kaplan-Meier plots on the right-hand side illustrate the cumulative risk for deficiency of other B group vitamins during follow-up for selected quantiles of the plurality-biomarker-score. Both plots serve to demonstrate that the risk is increasing non-linearly in the high end of the distribution of the plurality biomarker score. The plots are shown for the validation set part of the study population, i.e. 50% which was not included for derivation of the plurality-biomarker-scores (n=58 594 individuals).

FIG. 8b shows the hazard ratio of the same plurality-biomarker score with future onset of deficiency of other B group vitamins (ICD-10 code E53) when accounting for relevant risk factor characteristics of the study participants. The first two panels of results demonstrate that the risk prediction applications are consistent for men and women, as well as for people at different ages at time of blood sampling. The third panel shows that the magnitude of the hazard ratio is only modestly attenuated when accounting for body mass index and smoking status in the statistical modelling. The fourth panel shows that the predictive associations are strong both for lean individuals (BMI<25) as well as overweight or obese individuals (BMI>25). The last panel shows that the hazard ratio is substantially stronger when focusing on short-term risk of deficiency of other B group vitamins, here illustrated by using diagnoses recorded during the first 3 years after the blood samples were taken.

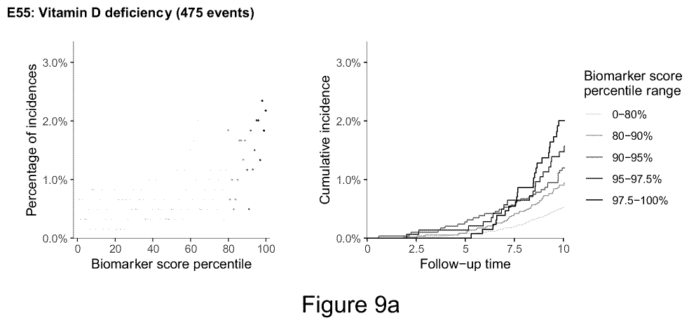

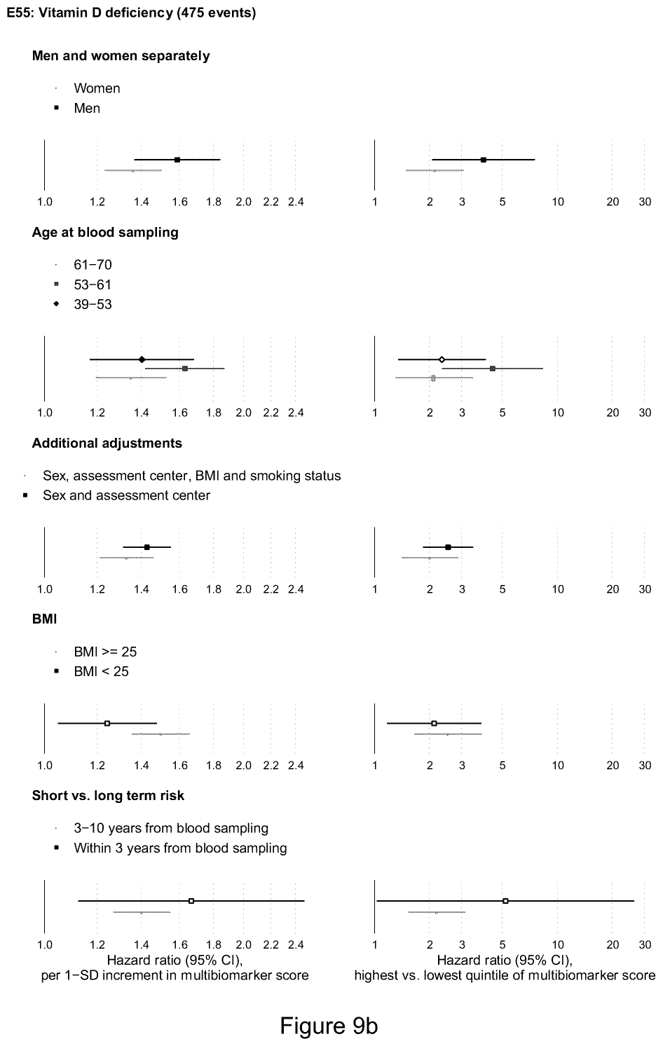

FIG. 9a shows the increase in risk for vitamin D deficiency (ICD-10 code E55) along with increasing levels of a biomarker risk score composed of the weighted sum of 20 biomarkers. On the left-hand side, the risk increase is plotted in the form of gradient percentile plots, showing the proportion of individuals who developed vitamin D deficiency during follow-up when binning individuals into the percentiles of the biomarker levels. Each dot corresponds to approximately 500 individuals. The Kaplan-Meier plots on the right-hand side illustrate the cumulative risk for vitamin D deficiency during follow-up for selected quantiles of the plurality-biomarker-score. Both plots serve to demonstrate that the risk is increasing non-linearly in the high end of the distribution of the plurality biomarker score. The plots are shown for the validation set part of the study population, i.e. 50% which was not included for derivation of the plurality-biomarker-scores (n=58 594 individuals).

FIG. 9b shows the hazard ratio of the same plurality-biomarker score with future onset of vitamin D deficiency (ICD-10 code E55) when accounting for relevant risk factor characteristics of the study participants. The first two panels of results demonstrate that the risk prediction applications are strong for men and women, as well as for people at different ages at time of blood sampling. The third panel shows that the magnitude of the hazard ratio is only modestly attenuated when accounting for body mass index and smoking status in the statistical modelling. The fourth panel shows that the predictive associations are strong both for lean individuals (BMI<25) as well as overweight or obese individuals (BMI>25). The last panel shows that the hazard ratio is substantially stronger when focusing on short-term risk of vitamin D deficiency, here illustrated by using diagnoses recorded during the first 3 years after the blood samples were taken.

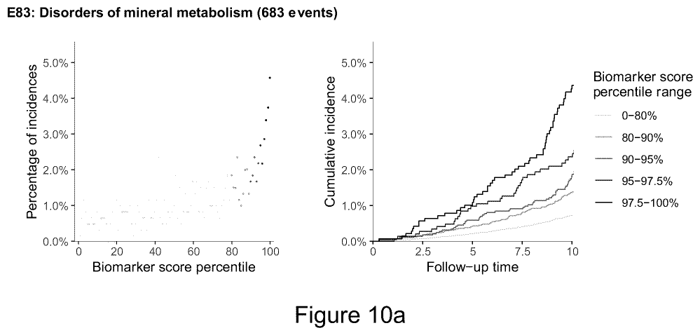

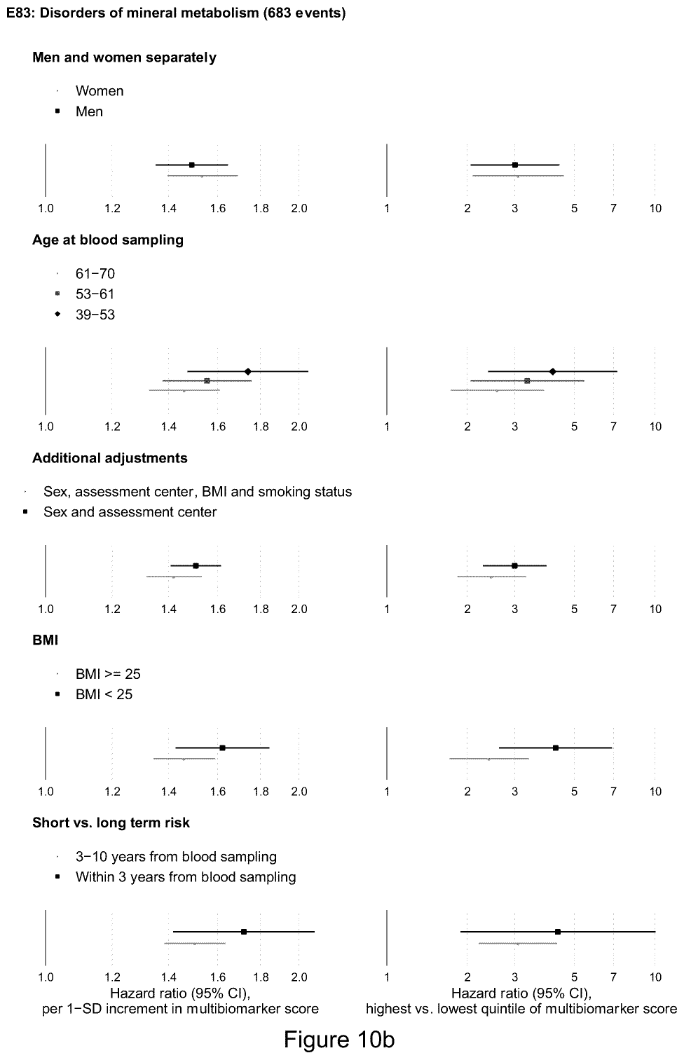

FIG. 10a shows the increase in risk for disorders of mineral metabolism (ICD-10 code E83) along with increasing levels of a biomarker risk score composed of the weighted sum of 20 biomarkers. On the left-hand side, the risk increase is plotted in the form of gradient percentile plots, showing the proportion of individuals who developed disorders of mineral metabolism during follow-up when binning individuals into the percentiles of the biomarker levels. Each dot corresponds to approximately 500 individuals. The Kaplan-Meier plots on the right-hand side illustrate the cumulative risk for disorders of mineral metabolism during follow-up for selected quantiles of the plurality-biomarker-score. Both plots serve to demonstrate that the risk is increasing non-linearly in the high end of the distribution of the plurality biomarker score. The plots are shown for the validation set part of the study population, i.e. 50% which was not included for derivation of the plurality-biomarker-scores (n=58 594 individuals).

FIG. 10b shows the hazard ratio of the same plurality-biomarker score with future onset of disorders of mineral metabolism (ICD-10 code E83) when accounting for relevant risk factor characteristics of the study participants. The first two panels of results demonstrate that the risk prediction applications are consistent for men and women, as well as for people at different ages at time of blood sampling. The third panel shows that the magnitude of the hazard ratio is only modestly attenuated when accounting for body mass index and smoking status in the statistical modelling. The fourth panel shows that the predictive associations are strong both for lean individuals (BMI<25) as well as overweight or obese individuals (BMI>25). The last panel shows that the hazard ratio is substantially stronger when focusing on short-term risk of disorders of mineral metabolism, here illustrated by using diagnoses recorded during the first 3 years after the blood samples were taken.

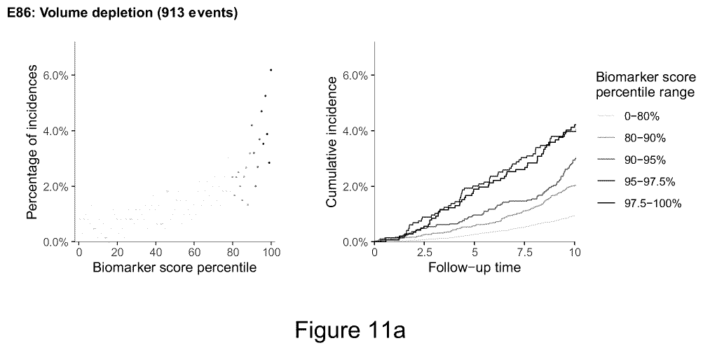

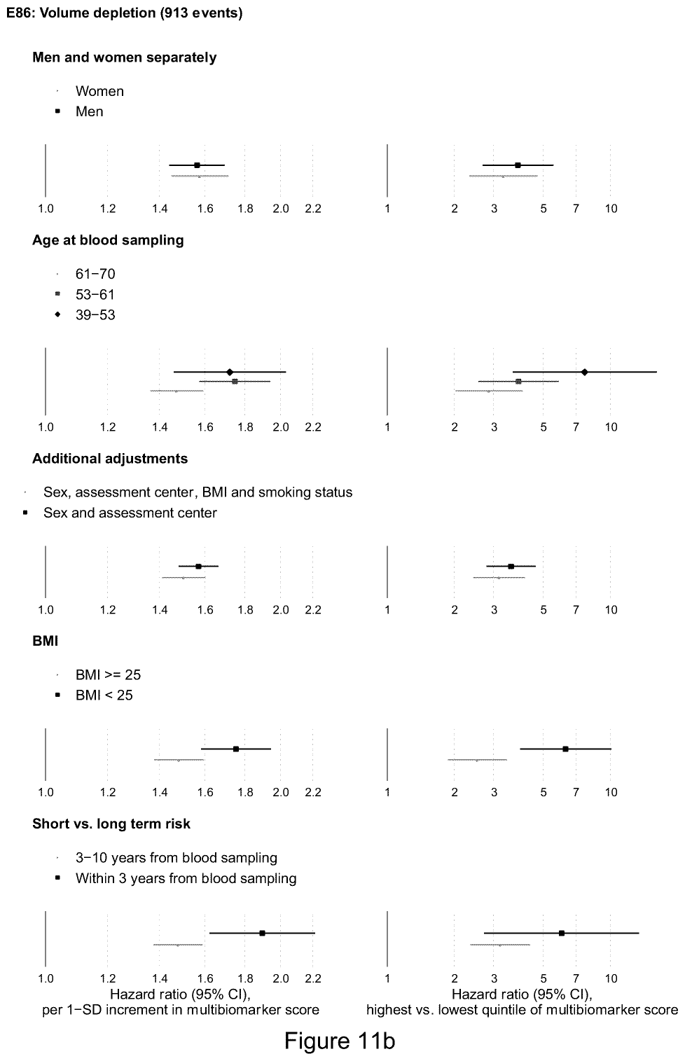

FIG. 11a shows the increase in risk for volume depletion (ICD-10 code E86) along with increasing levels of a biomarker risk score composed of the weighted sum of 20 biomarkers. On the left-hand side, the risk increase is plotted in the form of gradient percentile plots, showing the proportion of individuals who developed volume depletion during follow-up when binning individuals into the percentiles of the biomarker levels. Each dot corresponds to approximately 500 individuals. The Kaplan-Meier plots on the right-hand side illustrate the cumulative risk for volume depletion during follow-up for selected quantiles of the plurality-biomarker-score. Both plots serve to demonstrate that the risk is increasing non-linearly in the high end of the distribution of the plurality biomarker score. The plots are shown for the validation set part of the study population, i.e. 50% which was not included for derivation of the plurality-biomarker-scores (n=58 594 individuals).

FIG. 11b shows the hazard ratio of the same plurality-biomarker score with future onset of volume depletion (ICD-10 code E86) when accounting for relevant risk factor characteristics of the study participants. The first two panels of results demonstrate that the risk prediction applications are consistent for men and women, as well as for people at different ages at time of blood sampling. The third panel shows that the magnitude of the hazard ratio is only modestly attenuated when accounting for body mass index and smoking status in the statistical modelling. The fourth panel shows that the predictive associations are strong both for lean individuals (BMI<25) as well as overweight or obese individuals (BMI>25). The last panel shows that the hazard ratio is substantially stronger when focusing on short-term risk of volume depletion, here illustrated by using diagnoses recorded during the first 3 years after the blood samples were taken.

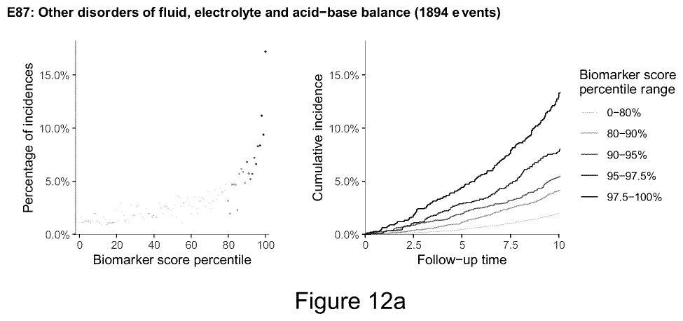

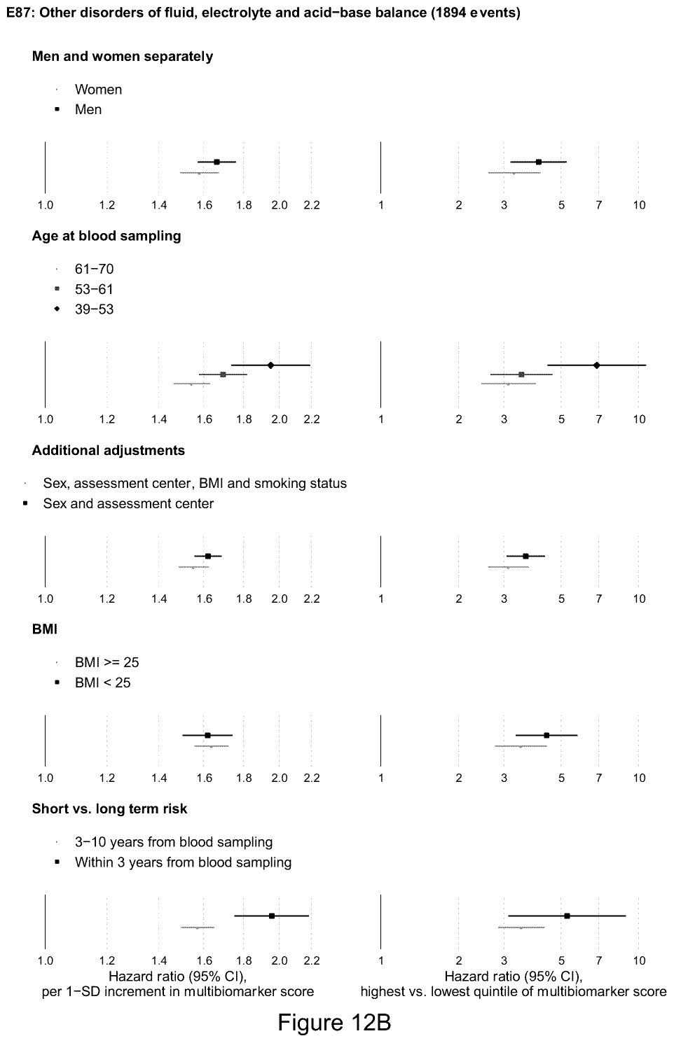

FIG. 12a shows the increase in risk for other disorders of fluid, electrolyte and acid-base balance (ICD-10 code E87) along with increasing levels of a biomarker risk score composed of the weighted sum of 20 biomarkers. On the left-hand side, the risk increase is plotted in the form of gradient percentile plots, showing the proportion of individuals who developed other disorders of fluid, electrolyte and acid-base balance during follow-up when binning individuals into the percentiles of the biomarker levels. Each dot corresponds to approximately 500 individuals. The Kaplan-Meier plots on the right-hand side illustrate the cumulative risk for other disorders of fluid, electrolyte and acid-base balance during follow-up for selected quantiles of the plurality-biomarker-score. Both plots serve to demonstrate that the risk is increasing non-linearly in the high end of the distribution of the plurality biomarker score. The plots are shown for the validation set part of the study population, i.e. 50% which was not included for derivation of the plurality-biomarker-scores (n=58 594 individuals).

FIG. 12b shows the hazard ratio of the same plurality-biomarker score with future onset of other disorders of fluid, electrolyte and acid-base balance (ICD-10 code E87) when accounting for relevant risk factor characteristics of the study participants. The first two panels of results demonstrate that the risk prediction applications are consistent for men and women, as well as for people at different ages at time of blood sampling. The third panel shows that the magnitude of the hazard ratio is only modestly attenuated when accounting for body mass index and smoking status in the statistical modelling. The fourth panel shows that the predictive associations are strong both for lean individuals (BMI<25) as well as overweight or obese individuals (BMI>25). The last panel shows that the hazard ratio is substantially stronger when focusing on short-term risk of other disorders of fluid, electrolyte and acid-base balance, here illustrated by using diagnoses recorded during the first 3 years after the blood samples were taken.

It is obvious to a person skilled in the art that with the advancement of technology, the basic idea may be implemented in various ways. The embodiments are thus not limited to the examples described above; instead they may vary within the scope of the claims.

The embodiments described hereinbefore may be used in any combination with each other. Several of the embodiments may be combined together to form a further embodiment. A method disclosed herein may comprise at least one of the embodiments described hereinbefore. It will be understood that the benefits and advantages described above may relate to one embodiment or may relate to several embodiments. The embodiments are not limited to those that solve any or all of the stated problems or those that have any or all of the stated benefits and advantages. It will further be understood that reference to ‘an’ item refers to one or more of those items. The term “comprising” is used in this specification to mean including the feature(s) or act(s) followed thereafter, without excluding the presence of one or more additional features or acts.

1. A method for determining whether a subject is at risk of developing a metabolic condition related to the nutritional state of the blood;
wherein the method comprises determining in a biological sample obtained from the subject a quantitative value of at least one biomarker of the following in the biological sample:
glycoprotein acetyls,
albumin,
ratio of docosahexaenoic acid to total fatty acids,
ratio of linoleic acid to total fatty acids,
ratio of monounsaturated fatty acids and/or of oleic acid to total fatty acids,
ratio of omega-3 fatty acids to total fatty acids,
ratio of omega-6 fatty acids to total fatty acids,
fatty acid degree of unsaturation,
docosahexaenoic acid,
linoleic acid,
omega-3 fatty acids,
omega-6 fatty acids,
citrate,
pyruvate,
alanine,
glutamine,
histidine,
leucine,
phenylalanine, and
valine; and
comparing the quantitative value(s) of the at least one biomarker to a control sample or to a control value;
wherein an increase or a decrease in the quantitative value(s) of the at least one biomarker, when compared to the control sample or to the control value, is/are indicative of the subject having an increased risk of developing the metabolic condition related to the nutritional state of the blood;
wherein the at least one biomarker comprises or is glycoprotein acetyls; and
wherein the metabolic condition related to the nutritional state of the blood comprises or is vitamin deficiency and/or disorder of mineral metabolism.

2. The method according to claim 1, wherein the method comprises determining in the biological sample quantitative values of two or more of the biomarkers.

3. The method according to claim 1, wherein the method comprises determining in the biological sample obtained from the subject a quantitative value of the following biomarkers:
glycoprotein acetyls; and
albumin; and
comparing the quantitative values of the biomarkers to a control sample or to a control value(s);
wherein an increase or a decrease in the quantitative values of the biomarkers, when compared to the control sample or to the control value, are indicative of the subject having an increased risk of developing the metabolic condition related to the nutritional state of the blood.

4. The method according to claim 1, wherein the method comprises determining in the biological sample obtained from the subject a quantitative value of the following biomarkers:
glycoprotein acetyls, and
at least one fatty acid measure(s) of the following: ratio of docosahexaenoic acid to total fatty acids, docosahexaenoic acid, ratio of linoleic acid to total fatty acids, linoleic acid, ratio of monounsaturated fatty acids and/or of oleic acid to total fatty acids, ratio of omega-3 fatty acids to total fatty acids, omega-3 fatty acids, ratio of omega-6 fatty acids to total fatty acids, omega-6 fatty acids, fatty acid degree of unsaturation; and
comparing the quantitative values of the biomarkers to a control sample or to a control values;
wherein an increase or a decrease in the quantitative values of the biomarkers, when compared to the control sample or to the control value, are indicative of the subject having an increased risk of developing the metabolic condition related to the nutritional state of the blood.

5. The method according to claim 1, wherein the metabolic condition related to the nutritional state comprises or is iron deficiency anemia, vitamin deficiency, and/or disorder of mineral metabolism.

6. The method according to claim 1, wherein the metabolic condition related to the nutritional state of the blood comprises or is iron deficiency anemia (D50); vitamin B12 deficiency anemia (D51); deficiency of other B group vitamins (E53); vitamin D deficiency (E55); deficiency of other nutrient elements (E61); and/or disorder of mineral metabolism (E83).

7. The method according to claim 1, wherein the metabolic condition related to the nutritional state and/or fluid balance of the blood comprises or is other iron deficiency anemia (D50.8); iron deficiency anemia, unspecified (D50.9); vitamin B12 deficiency anemia due to intrinsic factor deficiency (D51.0); disorder of phosphorus metabolism and/or phosphatases (E83.3); deficiency of other specified B group vitamins (E53.8; vitamin D deficiency, unspecified (E55.9); iron deficiency (E61.1); disorder of magnesium metabolism (E83.4); and/or disorder of calcium metabolism (E83.5).

8. The method according to claim 1, wherein the quantitative value of the at least one biomarker is/are measured using nuclear magnetic resonance spectroscopy.

9. The method according to claim 1, wherein the method further comprises determining whether the subject is at risk of developing the metabolic condition related to the nutritional state of the blood using a risk score, hazard ratio, odds ratio, and/or predicted absolute risk or relative risk calculated on the basis of the quantitative value(s) of the at least one biomarker or of the plurality of the biomarkers.

10. The method according to claim 9, wherein the risk score, hazard ratio, odds ratio, and/or predicted relative risk and/or absolute risk is calculated on the basis of at least one further measure.

11. The method according to claim 16, wherein the characteristic of the subject includes one or more of age, height, weight, body mass index, race or ethnic group, smoking, and/or family history of anemias and/or metabolic conditions related to the nutritional state of the blood.

12. The method according to claim 1, wherein the method comprises determining in the biological sample obtained from the subject a quantitative value of the following biomarkers:
glycoprotein acetyls,
albumin,
ratio of docosahexaenoic acid to total fatty acids,
ratio of linoleic acid to total fatty acids,
ratio of monounsaturated fatty acids and/or of oleic acid to total fatty acids,
ratio of omega-3 fatty acids to total fatty acids,
ratio of omega-6 fatty acids to total fatty acids,
fatty acid degree of unsaturation,
docosahexaenoic acid,
linoleic acid,
omega-3 fatty acids,
omega-6 fatty acids,
citrate,
pyruvate,
alanine,
glutamine,
histidine,
leucine,
phenylalanine, and
valine; and
comparing the quantitative values of the biomarkers to a control sample or to a control values;
wherein an increase or a decrease in the quantitative values of the biomarkers, when compared to the control sample or to the control value, are indicative of the subject having an increased risk of developing the metabolic condition related to the nutritional state of the blood.

13. The method according to claim 2, wherein the method comprises determining in the biological sample quantitative values of three or more of the biomarkers.

14. The method according to claim 13, wherein the method comprises determining in the biological sample quantitative values of four or more of the biomarkers.

15. The method according to claim 14, wherein the method comprises determining in the biological sample quantitative values of five or more of the biomarkers.

16. The method according to claim 10, wherein the at least one further measure is a characteristic of the subject.

17. The method according to claim 12, wherein the metabolic condition related to the nutritional state comprises or is iron deficiency anemia, vitamin deficiency, and/or disorder of mineral metabolism.

18. The method according to claim 12, wherein the metabolic condition related to the nutritional state of the blood comprises or is iron deficiency anemia (D50); vitamin B12 deficiency anemia (D51); deficiency of other B group vitamins (E53); vitamin D deficiency (E55); deficiency of other nutrient elements (E61); and/or disorder of mineral metabolism (E83).

19. The method according to claim 12, wherein the metabolic condition related to the nutritional state and/or fluid balance of the blood comprises or is other iron deficiency anemia (D50.8); iron deficiency anemia, unspecified (D50.9); vitamin B12 deficiency anemia due to intrinsic factor deficiency (D51.0); disorder of phosphorus metabolism and/or phosphatases (E83.3); deficiency of other specified B group vitamins (E53.8; vitamin D deficiency, unspecified (E55.9); iron deficiency (E61.1); disorder of magnesium metabolism (E83.4); and/or disorder of calcium metabolism (E83.5).

20. The method according to claim 12, wherein the method further comprises determining whether the subject is at risk of developing the metabolic condition related to the nutritional state of the blood using a risk score, hazard ratio, odds ratio, and/or predicted absolute risk or relative risk calculated on the basis of the quantitative value(s) of the at least one biomarker or of the plurality of the biomarkers.
